(19) United States
(12) Patent Application Publication
(10) Pub. No.: US 2015/0359815 A1
(43) Pub. Date: Dec. 17, 2015

(54) PAK1 INHIBITION FOR TREATMENT OF ACUTE MYELOID LEUKEMIA AND MYELODYSPLASTIC SYNDROMES

(71) Applicant: ALBERT EINSTEIN COLLEGE OF MEDICINE OF YESHIVA UNIVERSITY, BRONX, NY (US)

(72) Inventors: ULRICH G. STEIDL, NEW ROCHELLE, NY (US); ASHLEY M. PANDOLFI, NEW YORK, NY (US); ROBERT F. STANLEY, NEW YORK, NY (US)

(73) Assignee: ALBERT EINSTEIN COLLEGE OF MEDICINE OF YESHIVA UNIVERSITY, BRONX, NY (US)

(21) Appl. No.: 14/845,361

(22) Filed: Sep. 4, 2015

Related U.S. Application Data
(63) Continuation-in-part of application No. PCT/US2014/024239, filed on Mar. 12, 2014.
(60) Provisional application No. 61/789,345, filed on Mar. 15, 2013.

(51) Int. Cl. A61K 31/7105; A61K 31/519; C12Q 1/68; A61K 31/105

(52) U.S. Cl. CPC A61K 31/7105; A61K 31/105; A61K 31/519; C12Q 1/6886; C12Q 2600/178; C12Q 2600/158; C12Q 2600/136

(57) ABSTRACT

Methods are disclosed for treating acute myeloid leukemia (AML) and myelodysplastic syndromes (MDS) using inhibition of p21 protein (Cdc42/Rac)-activated kinase (PAK1).

### CROSS REFERENCE TO RELATED APPLICATIONS

This application is a continuation-in-part of and claims priority of PCT International Patent Application No. PCT/U.S. 2014/024239, filed Mar. 12, 2014, which designates the United States of America and which claims the benefit of U.S. Provisional Patent Application No. 61/789,345, filed on Mar. 15, 2013, the contents of which are herein incorporated by reference in their entirety.

### BACKGROUND OF THE INVENTION

Throughout this application various publications are referred to in parenthesis. Full citations for these references may be found at the end of the specification. The disclosures of these publications, and of all patents, patent application publications and books referred to herein, are hereby incorporated by reference in their entirety into the subject application to more fully describe the art to which the subject invention pertains.

Acute myeloid leukemia (AML) and myelodysplastic syndromes (MDS) are heterogeneous clonal neoplastic diseases that originate from transformed cells that have progressively acquired critical genetic changes that disrupt key differentiation- and growth-regulatory pathways (Hanahan and Weinberg, 2000; Marcucci et al., 2011). Less than one third of AML patients achieve durable remission with current treatment regimens, and prognostication and risk stratification of individual patients remains very challenging, in particular in favorable and standard risk groups.

Analysis of pre-leukemic hematopoietic stem and progenitor cells (HSPC) in a marine model of AML revealed the non-clustered H2.0-like homeobox (Hlx) gene to be 4-fold upregulated compared to wild-type (WT) HSPC (Steidl et al., 2006) suggesting that Hlx may be involved in malignant transformation. HLX is the highly conserved human/murine homologue of the homeobox gene H2.0, which shows tissue-specific expression throughout development in Drosophila melanogaster (Allen et al, 1991; Hentsch et al 1996). Additional studies two decades ago detected HLX expression in hematopoietic progenitors and in leukemic blasts of patients with AML, and a study of HLX-deficient fetal liver cells suggested a decrease of colony-formation capacity (Deguchi and Kehrl, 1991; Deguchi et al., 1992).

The present invention addresses the need for treatments for acute myeloid leukemia (AML) and myelodysplastic syndromes (MDS) as well as tumors expressing elevated levels of HLX.

### SUMMARY OF THE INVENTION

The invention provides methods of treating acute myeloid leukemia (AML), myelodysplastic syndrome (MDS), or a tumor having elevated expression of 1-12.0-like homeobox (HLX) and/or elevated expression of p21 protein (Cdc42/Rac)-activated kinase (PAK1) in a subject, the methods comprising administering to the subject a PAK1 inhibitor in an amount effective to treat AML, MDS or the tumor having elevated expression of HLX and/or PAK1.

The invention also provides methods for screening for a candidate compound for treating a subject with acute myeloid leukemia (AML), myelodysplastic syndrome (MDS), andlor a tumor having elevated expression of 1-12.0-like homeobox (HLX) andlor elevated expression of p21 protein (Cdc42/Rac)-activated kinase (PAK1), the methods comprising determining whether or not the compound inhibits PAK1, wherein a compound that inhibits PAK1 is a candidate compound for treating a subject with AML, MDS, and/or a tumor having elevated expression of HLX and/or elevated expression of PAK1.

### BRIEF DESCRIPTION OF THE DRAWINGS

FIG. 1A-1C. Knockdown of PAK1 by short hairpin (sh) RNA in KG1a AML cells led to a significant inhibition of cell proliferation (p<0.05) (A) and clonogenicity (p<0.03) (B). Error bars indicate SD (N=4). (C) mRNA expression of PAK1 gene upon knockdown in KG1a cells. Relative expression changes compared to control-transfected cells as measured by quantitative real-time PCR.

FIG. 2. 1,1′-Disulfanediyldinaphthalen-2-ol (“Compound 1”) inhibits PAK I and reduces proliferation of AML cells as demonstrated. in four different AML cell lines (HL60, THP1, KG1a and MOLM13).

FIG. 3A-3B. An inactive control analog of Compound 1 is not effective in reducing proliferation of HL60 AML cells. (A) Reduction of proliferation of HL60 AML cells by Compound 1. (B) Lack of reduction of proliferation of HL60 AMIL cells by an inactive analog of Compound 1.

FIG. 4. Inhibition of PAK1 by Compound 1 reduces colony formation ability of AML cells.

FIG. 5. Compound 1-mediated inhibition of PAK1 has a greater effect on growth of AML and MDS cells isolated from the bone marrow of AML and MDS patients than on healthy hematopoietic cells from the bone marrow of healthy donors. *p<0.05, **p<0.01.

FIG. 6. Inhibition of PAK1 by Compound 1 induces apoptosis in AML cells. 24 hours of incubation with Compound 1 (assay by Annexin V/DAPI), Black—Percentage of live cells; Grey—Percentage of apoptotic cells.

FIG. 7. Inhibition of PAK1 by (S)-N-(2-(dimethylamino)-1-phenylethyl)-6,6-dimethyl-3-((2-methylthieno[3,2-d]pyrimidin-4-yl)amino)-4,6-dihydropyrrolo[3,4-c]pyrazole-5(1H)-carboxamide (“Compound 2”) reduces proliferation of (THP AIVIL cells.

FIG. 8. Compound 1-mediated inhibition of PAK1: effects on colony formation of cells from a patient with myelodysplastic syndrome (MDS). Inhibition of PAKI is effective in both myelodysplastic syndrome (MDS) and acute myeloid leukemia (AML).

FIG. 9. Effect of Compound 1 (IPA-3) on downstream signaling pathways of PAKI. Measurement of phosphorylation status of kinases in THPI leukemia cells after 4 hours of treatment with 6 μg of IPA-3 or DMSO demonstrates on-target effects of Compound 1 (Akt 1/2/3 S473) and that several additional oncogenic signaling pathways are significantly inhibited in AML cells in response to PAK1 inhibition.

FIG. 10A-10B. STAT4 expression is increased upon treatment with Compound 1 (IPA-3) or PAKI shRNA knockdown in THP1 cells. A) Western Blot; B) qRT-PCR.

FIG. 11. Patients with myelodysplastic syndromes (MDS) show significantly decreased overall survival when they express high PAK1 levels, in comparison to MDS patients with low PAK1 levels.

FIG. 12A-12B, Evaluation of PAK1 expression reduction in THP1 AML cells by shRNAs, A) Evaluated mRNA levels by RT-gPCR. B) Significant prolongation of survival of mice injected with 300,000 THP1 cells expressing either a shRNA #1 or shRNA #2 to PAK1 compared to THP expressing a non-silencing control shRNA.

### DETAILED DESCRIPTION OF THE INVENTION

The invention provides a method of treating acute myeloid leukemia (AML), myelodysplastic syndrome (MDS), or a tumor having elevated expression of H2.0-like homeobox (HLX) and/or elevated expression of p21 protein (Cdc42/Rac)-activated kinase (PAK1) in a subject, the method comprising administering to the subject a PAK1. inhibitor in an amount effective to treat AML, MDS or the tumor having elevated expression of HLX and/or PAK1.

In different embodiments, for example, the subject has AML or the subject has MDS. The subject may have elevated expression of HLX. The subject can have, for example, a tumor having elevated expression of HLX or a tumor having elevated expression of HLX and elevated expression of PAK1. In other embodiments, the subject can have a tumor where the activity of PAK1 is increased.

As used herein, elevated expression of HLX and elevated expression of PAK1 means a level that is elevated compared to the level of HLX or PAK1 in a subject who does not have AML, MDS, or a cancer. Similarly, increased PAK1 activity means that the activity of PAK1 is increased compared to the activity level in a subject who does not have AML, MDS, or a cancer.

Examples of cell types for testing PAK1 expression and activity and HLX expression include but are not limited to: 1) tumor bulk cells “blast cells” of an AML or MDS patient, 2) total mononuclear cells from the blood or marrow of an AML or MDS patient, andior 3) leukemic stem cells of an AML or MDS patient. Controls could include, but are not limited to, for example: total mononuclear cells from the blood or marrow of a healthy donor, CD34 enriched cells from a healthy donor, and/or hematopoietic stem cells from a healthy donor.

In different embodiments, the PAK1 inhibitor can be, for example, a pyrrole pyrazole compound or a pyrrolo[2,3-b]pyridine derivative. Preferred PAK1 inhibitors include 1,1′-Disulfanediyldinaphthalen-2-ol and (S)-N-(2-(dimethylamino)-1-phenylethyl)-6,6-dimethyl-3-((2-methylthieno[3,2-d]pyrimidin-4-yl)amino)-4,6-dihydropyrrolo[3,4-c]pyrazole-5(1H)-carboxamide. In Examples below, 1,1′-Disulfanediyldinaphthalen-2-ol is referred to as “Compound 1” and (S)—N-(2-(dimethylamino)-1-phenylethyl)-6,6-dimethyl-3-((2-methylthieno[3,2-d]pyrimidin-4-yl)amino)-4,6-dihydropyrrolo[3,4-c]pyrazole-5(1H)-carboxamide is referred to as “Compound 2.” Compound 1 can be obtained from EMD Millipore (emdmillipore.com) (506106, p21-Activated Kinase Inhibitor III, IPA-3). Compound 2 can be obtained from Active Biochem (activebiocherucom) (Product Name:PF-03758309, CAT#: A-1091). Other PAK inhibitors have been described in, for example, U.S. Pat. Nos. 7,888,508 and 8,067,591, the contents of which are herein incorporated by reference.

The inhibitor can be, for example, a small organic molecule of less than 2000 daltons, or an antibody or antibody fragment directed against PAK1. The inhibitor can also be, for example, an antisense oligonucleotide, aptamer, or peptide interfering with PAK1 protein function.

The PAK 1 inhibitor can also be a short hairpin nucleic acid. The short hairpin sequences silence PAK1 gene expression via RNA interference. Examples of short hairpin nucleic acid sequences for human PAK 1 include CGATGAGAAATACCAGCACTA (SEQ ID NO:31) and GCGATCCTAAGAAGAAATATA (SEQ ID NO:32). These are DNA sequences which can be expressed by lentiviruses. Cells are infected with these viruses, and then the cells transcribe the viral DNA into the actual shRNAs.

Preferably, the PAK1 inhibitor reduces proliferation of AML, MDS and/or tumor cells having elevated expression of HLX and/or PAK 1. Preferably, the PAK 1 inhibitor induces apoptosis in AML, MDS and/or tumor cells having elevated expression of FILX and/or PAK1. Preferably, the PAK1 inhibitor reduces colony formation of AML, MDS and/or tumor cells having elevated expression of HLX and/or PAK1.

Preferably, treatment of the subject with the PAK1 inhibitor increases survival of the subject compared to untreated control subjects.

The invention also provides a method for screening for a candidate compound for treating a subject with acute myeloid leukemia (AML), myelodysplastic syndrome (MDS), and/or a tumor having elevated expression of H2.0-like homeobox (HLX) and/or elevated expression of p21 protein (Cdc42/Rac)-activated kinase (PAK1), the method comprising deteimining whether or not the compound inhibits PAK1, wherein a compound that inhibits PAK1 is a candidate compound for treating a subject with AML, MDS, and/or a tumor having elevated expression of HLX and/or elevated expression of PAK 1.

The screening method can be carried out, for example, using an AML cell line, such as HL60, THP1, KG1a or MOLMI3. Inhibition of PAK1 can be detected, for example, by decrease of PAK1 (mRNA or protein) expression, increased expression of Signal Transducer and Activator of Transcription protein 4 (STAT4) in the AML cells, and/or by decrease of AKT1/2/3 serine 473 phosphorylation and/or STAT3 phosphorylation.

As used herein, HLX gene is a human gene encoding H2.0-like homeobox protein. (Convention has upper case “HLX” as the human gene and “Hlx” as non-human equivalents).

The HLX gene has RefSeq Accession no. NM_{—}0219583.

| | |
|---|---|
| 1 | aaaactttgg gagtttttag agacgagttt tttttttttt ctattacttt tccccccccc |
| 61 | taactaacgg actattattg ttgttgtttt aaatttagct cttagggctt agctatttgg |
| 121 | gttttcttgc ggtgtccggc tcccgtctcc ctggctcccc cgcccgccct gcggccccag |
| 181 | cgcccctcgc tctcatccag cccgcgagga gtgcgggcgc cgcgccgcct ttaaagcgag |
| 241 | gccagggagc gaggcggtga ccggccgaga tccggccctc gcctcctccc tcggtggcgc |
| 301 | tagggctccc ggcctctctt cctcagtgcg ggcggagaag cgaaagcgga tcgtcctcgg |
| 361 | ctgccgccgc cttctccggg actcgcgcgc ccctccccgc gcgcccaccc acccagtccg |
| 421 | gctggactgc ggcagccgcg cggctcaccc cggcaggatg ttcgcagccg ggctggctcc |
| 481 | cttctacgcc tccaacttca gcctctggtc ggccgcttac tgctcctcgg ccggcccagg |
| 541 | cggctgctcc ttccccttgg accccgccgc cgtcaaaaag ccctccttct gcatcgcaga |
| 601 | cattctgcac gccggcgtgg gggatctggg ggcggccccg gagggcctgg caggggcctc |
| 661 | ggccgccgcc ctcaccgcgc acttgggctc ggttcacccg cacgcctctt tccaagcggc |
| 721 | ggccagatcc ccgcttcgac ccaccccagt ggtggcgccc tccgaagtcc cggctggctt |
| 781 | cccgcagcgg ctgtctccgc tctcagccgc ctaccaccac catcacccgc aacaacaaca |
| 841 | gcagcagcaa cagccgcagc agcaacagcc tccgcctccg ccccgggctg gcgccctgca |
| 901 | gcccccggcc tcggggacgc gagtggttcc gaacccccac cacagtggct ctgccccggc |
| 961 | cccctccagc aaagacctca aatttggaat tgaccgcatt ttatctgcag aatttgaccc |
| 1021 | aaaagccaaa gaaggcaaca cgctgagaga tctcacttcc ctgctaaccg gtgggcggcc |
| 1081 | cgccggggtg cacctctcag gcctgcagcc ctcggccggc cagctcttcg catctccaga |
| 1141 | tcccattaac gaggcttctg caatcctgag tcccttaaac tcgaacccaa gaaattcagt |
| 1201 | tcagcatcag tcccaagaca cgtttccagg tccctatgct gtgctcacga aggacaccat |
| 1261 | gccgcagacg tacaaaagga agcgttcatg gtcgcgcgct gtgttctcca acctgcagag |
| 1321 | gaaaggcctg gagaaaaggt ttgagattca gaagtacgtg accaagccgg accgaaagca |
| 1381 | gctggcggcg atgctgggcc tcacggacgc acaggtgaag gtgtggttcc agaaccggcg |
| 1441 | gatgaagtgg cggcactcca aggaggccca ggcccaaaag gacaaggaca aggaggctgg |
| 1501 | cgagaagcca tcaggtggag ccccggctgc ggatggcgag caggacgaga ggagccccag |
| 1561 | ccgttctgaa ggcgaggctg agagcgagag cagcgactcc gagtccctgg acatggcccc |
| 1621 | cagcgacacg gagcggactg aggggagcga gcgttctctg caccaaacaa cagttattaa |
| 1681 | ggccccggtc actggcgccc tcattaccgc cagcagtgct gggagtggtg ggagcagcgg |
| 1741 | cggcggcggc aatagtttca gcttcagcag cgccagcagt cttagtagca gcagcaccag |
| 1801 | tgcgggttgc gccagcagcc ttggcggcgg cggcgcctcg gagcttctcc ctgcaacaca |
| 1861 | gcccacagcc agcagcgctc ccaaaagccc cgagccagcc caaggcgcgc ttggctgctt |
| 1921 | atagactgta ctagggcgga ggggatccgg gccttgcgtg cagcctccca accatgggct |
| 1981 | gggttttgtg cttactgtat gttggcgact tggtagggca ggagacgcag cgtggagcct |
| 2041 | acctcccgac attcacgctt cgccccacgc tgctccgact ggctgcagcg gacactgccc |
| 2101 | aaagcagagg ggagtctcag cgtcctgcta gccagccgaa cacttctctc cggaagcagg |
| 2161 | ctggttcgac tgtgaggtgt ttgactaaac tgtttctctg actcgcccca gaggtcgcgg |
| 2221 | ctcaaaggca cttaggacgc cttaaatttg taaacaaaat gtttactacg gtttgtaaaa |
| 2251 | aaaaaaaaaa aaaaaaaaaa aaaaaaaa |

(NCBI Reference Sequence: NM_{—}021958.3; SEQ ID NO:33). In an embodiment, each t in the above sequence is replaced with a u.

Human HLX has the amino acid sequence (NCBI Reference Sequence: NP_{—}068777.1, SEQ ID NO:34):

| | |
|---|---|
| 1 | mfaaglapfy asnfslwsaa ycssagpggc sfpldpaavk kpsfciadil hagvgdlgaa |
| 61 | peglagasaa aitahlgsvh phasfqaaar splrptpvva psevpagfpq rlsplsaayh |
| 121 | hhhpqqqqqq qqpqqqqppp ppragalqpp asgtrvvpnp hhsgsapaps skdlkfgidr |
| 181 | ilsaefdpkv kegntlrdlt slltggrpag vhlsglqpsa gqffasldpi neasailspl |
| 241 | nsnprnsvqh qfqdtfpgpy avltkdtmpq tykrkrswsr avfsnlqrkg lekrfeiqky |
| 301 | vtkpdrkqla amlgltdaqv kvwfqnrrmk wrhskeaqaq kdkdkeagek psggapaadg |
| 361 | eqderspsrs egeaesessd sesldmapsd tertegsers lhqttvikap vtgalitass |
| 421 | agsggssggg gnsfsfssas slsssstsag casslgggga sellpatqpt assapkspep |
| 481 | aggalgcl. |

PAK1 is p21 protein (Cdc42/Rac)-activated kinase (a serine/threonine-protein kinase enzyme) that in humans is encoded by the PAK1 gene. Human PAK1 has the amino acid sequence (GenBank: AAI09300.1, SEQ ID NO:35):

| | |
|---|---|
| 1 | msnngldiqd kppappmrnt stmigagskd agtlnhgskp lppnpeekkk kdrfyrsilp |
| 61 | gdktnkkkek erpeislpsd fehtihvgfd avtgeftgmp eqwarllqts nitkseqkkn |
| 121 | pgavldvlef ynskktsnsq kymsftdksa edynssnaln vkavsetpav ppvsededdd |
| 181 | dddatpppvi aprpehtksv ytrsvieplp vtptrdvats pisptenntt ppdaltrnte |
| 241 | kqkkkpkmsd eeileklrsi vsvgdpkkky trfekigqqa sgtvytamdv atgqevaikq |
| 301 | mnlqqqpkke liineilvmr enknpnivny ldsylvgdel wvvmeylagg sltdvvtetc |
| 361 | mdegqiaavc reclqalefl hsnqvihrdi ksdnillgmd gsvkltdfgf cagitpeqsk |
| 421 | rstmvgtpyw mapevvtrka ygpkvdiwsl gimaiemieg eppylnenpl ralyliatng |
| 481 | tpelqnpekl saifrdflnr clemdvekrg sakellqhqf lkiakplssl tpliaaakea |
| 541 | tknnh. |

In an embodiment, the agents of the invention as described herein are administered in the form of a composition comprising the agent and a carrier. The term “carrier” is used in accordance with its art-understood meaning, to refer to a material that is included in a pharmaceutical composition but does not abrogate the biological activity of pharmaceutically active agent(s) that are also included within the composition. Typically, carriers have very low toxicity to the animal to which such compositions are to be administered. In some embodiments, carriers are inert.

In one embodiment of the methods, the HLX or PAK1 expression level or activity level of the gene product thereof is detected using a detectable agent. The detectable agent can be an antibody or a fragment of an antibody, which is itself detectable, e.g. by a secondary antibody, or which is labeled with a detectable marker such as a radioisotope, a fluorophore, a dye etc. permitting detection of the presence of the bound agent by the appropriate machine, or optionally in the case of visually detectable agents, with the human eye. In an embodiment, the amount of detectable agent can be quantified.

Acute myeloid leukemia (AML) is a cancer of the myeloid line of blood cells, characterized by the rapid growth of abnormal white blood cells that accumulate in the bone marrow and interfere with the production of normal blood cells.

The myelodysplastic syndromes (MDS, formerly known as preleukemia) are a collection of hematological conditions that involve ineffective production (or dysplasia) of the myeloid class of blood cells. Patients with MDS often develop severe anemia and require frequent blood transfusions. In most cases, the disease worsens and the patient develops cytopenias (low blood counts) due to progressive bone marrow failure. In about one third of patients with MDS, the disease transforms into acute myelogenous leukemia (AML), usually within months to a few years. The myelodysplastic syndromes are all disorders of the stem cell in the bone marrow.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Example 1—Role of HLX and PAK1 in Acute Myeloid Leukemia

### Summary

Homeobox domain-containing transcription factors are important regulators of hematopoiesis. Increased levels of non-clustered H2.0-like homeobox (HLX) lead to loss of functional hematopoietic stem cells and formation of aberrant progenitors with unlimited serial clonogenicity and blocked differentiation. Inhibition of HLX reduces proliferation and clonogenicity of leukemia cells, overcomes the differentiation block, and leads to prolonged survival. HLX regulates a transcriptional program, including PAK1 and BTG1, that controls cellular differentiation and proliferation. HLX is overexpressed in 87% of patients with acute myeloid leukemia (AML) and independently correlates with inferior overall survival (N=601, p=2.3×10−6). HLX is a key regulator in immature hematopoietic and leukemia cells, and a prognostic marker and therapeutic target in AML. This study was first reported in Kawahara et al. (2012).

### Materials and Methods

Mice and cells. FVB/nJ mice (Ly5.1), C57BLI6J (Ly5.2) mice, and B6.SJL-Ptprca Pepcb/BoyJ (Pep boy, Ly5.1), and NOD.Cg-Prkdcscid Il2rgtmlWjl/Sz1 (NSG) mice were used for in vitro assays and in vivo transplantation assays. Mice with targeted disruption of the upstream regulatory element (LIRE) of the PU.1 gene have been previously described (Rosenbauer,Wagner, et al, 2004). All animal experiments were approved by the Institutional Animal Care and Use Committee of the Albert Einstein College of Medicine (protocol # 20080109). URE cells were established and maintained as described previously (Steidl,Rosenbauer, et al, 2006). Human AML cell lines THP1, MOLM13, and KG1a were cultured under standard conditions.

Flow Cytometric Analysis and Sorting. Mononuclear cells were purified by lysis of erythrocytes. For analysis and sorting, antibodies directed against CD4[GK1.5], CD8a[53-6.7], CD 19[eBio1D3], Gr-1[RB6-8C5], B220[RA3-6B2], F4/80[BM8], c-kit[ACK2], Sca-1[D7], CD34[RAM34], CD16/32[93], Flk-2[A2F10], Mac1[M1/70], Ter119[TER-119], and Thy-1.2[53-2-1] were used. To distinguish donor from host cells in transplanted mice, cells were additionally stained with anti-CD45.1[A20] and CD45.2[104]. Analysis and sorting were performed using a FACSAria II Special Order System (BD Biosciences, San Jose, CA). For sorting Lin-Kit+ cells for in vivo assays, PE-Cy5-conjugated CD4, CD8a, CD19, B220, Ter119, and Gr-1 (anti-lineage) antibodies, and APC-conjugated c-kit antibody were used. For analyzing hematopoietic stem and early progenitor cells in vivo, PE-conjugated Ly5.2 antibody, pacific blue-conjugated Sca-1 antibody, PE-Cy7-conjugated Thy1.2 antibody, and biotin-conjugated Flk-2 antibody followed by APC-AlexaFluor 750 conjugated streptavidin were used. For analyzing committed progenitors, APC-conjugated Ly5.2 antibody, PE-Cy5-conjugated anti-lineage antibodies, APC-AlexaFluor 780-conjugated c-kit antibody, pacific blue-conjugated Sca-1 antibody, PE-conjugated Fc□:RH/III antibody, and biotin-conjugated CD34 antibody followed by PE-Cy7-conjugated streptavidin were used. For differentiation studies, PE-conjugated Gr-1 antibody, AFC-conjugated Mac 1 antibody, eFluorTM450 conjugated F4/80 antibody, and APC-AlexaFluor 780-conjugated c-kit antibody were used. For analysis of additional cell surface markers, biotin-conjugated CD49b, APC-conjugated CD11b, PE-conjugated CD44, APC-conjugated CD11c, PE-conjugated CD150, and biotin-conjugated FcγRII/III antibodies were used.

Lentiviral vectors and transduction. For overexpression studies, the mouse Hlx coding sequence was introduced into the EcoRI site of a pCAD-IRES-GFP lentiviral construct (Steidl, Steidl, et al, 2007). For BTG1 overexpression, the human BTG I coding sequence was introduced into the EcoRI site of the pCAD-TIRES-GFP construct. For murine knockdown studies, shRNA template oligonucleotides (target sense strand-loop-target antisense strand-TTTTT) were inserted into the pSIHI-H1-copGFP shRNA vector (System Biosciences, Mountain View, Calif.). For human knockdown studies, the pGIPZ system from Open Biosystems, Huntsville, Ala. was utilized. For knockdown of PAK1, the plko.1-puro vector system (Sigma) with non-silencing control or human PAK1 target was utilized. Lentiviral particles were produced utilizing 293T cells, and concentrated by ultracentrifugation. For overexpression studies, sorted LinKit+ cells from WT C57BL/6J (Ly5.2) BM (for in vivo assays) or LinKit+Sca-1+ cells from WT FVB/nJ BM (for in vitro assays) were treated with control virus or Hlx virus. In brief_{;} sorted cells were cultured with lentiviral supernatants in the presence of 8 μg/ml olybrene. 24 hours after transduction, cells were washed with PBS and then used for experiments. After 40 hours, transduction efficiency was detei mined by flow-cytometry. For knockdown studies, cells were incubated with short-hairpin-containing lentivirus for 24 hours. GFP+ cells were sorted using a FACS Aria II sorter (BD Biosciences) and used for experiments.

Quantitative real-time PCR. Total RNA was extracted from FACS-sorted cells or cultured cells using RNeasy Micro kit (Qiagen, Valencia, Calif.). cDNA was synthesized by Superscript II reverse transcriptase (Invitrogen, Carlsbad, Calif.). Real-time PCR was performed using an iQ5 real-time PCR detection system (BIO-RAD, Hercules, Calif.) with 1 cycle of 50° C. (2 min) and 95° C. (10 min) followed by 40 cycles of 95° C. (15 sec) and 60° C. (1 min) using Power SYBR Green PCR master mix (AB, Carlsbad, Calif.).

Primer sequences used were as follows (FW=forward, RV=reverse):

| (SEQ ID NO: 1) | |
|---|---|
| mouse Hlx FW | TTCAGCATCAATTCCAAGACACA |
| (SEQ ID NO: 2) | |
| mouse Hlx RV | ACCTCTTCTCCAGGCCTTTTCT |
| (SEQ ID NO: 3) | |
| mouse Btg1 FW | TCATCTCCAAGTTCCTCCGCAC |
| (SEQ ID NO: 4) | |
| mouse Btg1 RV | CAACGGTAACCTGATCCCTTGC |
| (SEQ ID NO: 5) | |
| mouse FoxO4 FW | TCTACGAATGGATGGTCCGCAC |
| (SEQ ID NO: 6) | |
| mouse FoxO4 RV | CTTGCTGTGCAAGGACAGGTTG |
| (SEQ ID NO: 7) | |
| mouse Gadd45a FW | CCTGGAGGAAGTGCTCAGCAAG |
| (SEQ ID NO: 8) | |
| mouse Gadd45a RV | GTCGTCTTCGTCAGCAGCCAG |
| (SEQ ID NO: 9) | |
| mouse Hdac7 FW | CGCCTCAAACTGGATAACGGGA |
| (SEQ ID NO: 10) | |
| mouse Hdac7 RV | GCATTGGAGGAATGCAGCTCGT |
| (SEQ ID NO: 11) | |
| mouse Pak1 FW | ATTGCTCCACGCCCAGAACACA |
| (SEQ ID NO: 12) | |
| mouse Pak1 RV | AAGCATCTGGCGGAGTGGTGTT |
| (SEQ ID NO: 13) | |
| mouse Gapdh FW | CCAGCCTCGTCCCGTAGAC |
| (SEQ ID NO: 14) | |
| mouse Gapdh RV | GCCTTGACTGTGCCGTTGA |
| (SEQ ID NO: 15) | |
| mouse Trp63 FW | TGAGCCGTGAGTTCAATGAG |
| (SEQ ID NO: 16) | |
| mouse Trp63 RV | ACCTGTGGTGGCTCATAAGG |
| (SEQ ID NO: 17) | |
| mouse RhoB FW | TATTTTCGGAGGCAGAATGG |
| (SEQ ID NO: 18) | |
| mouse RhoB RV | GATGGCCAAGTCCAGTTCAT |
| (SEQ ID NO: 19) | |
| mouse Zfp361 FW | CCCCTACCTTGGACAACTCA |
| (SEQ ID NO: 20) | |
| mouse Zfp361 RV | AAGAGATGGGGTGGAGAGGT |
| (SEQ ID NO: 21) | |
| mouse Fyn FW | GGAAAAAGGATCCGGAAGAG |
| (SEQ ID NO: 22) | |
| mouse Fyn RV | GCAGGCTCTCACAGGTTTTC |
| (SEQ ID NO: 23) | |
| human PAK1 FW | GTGAAGGCTGTGTCTGAGACTC |
| (SEQ ID NO: 24) | |
| human PAK1 RV | GGAAGTGGTTCAATCACAGACCG |
| (SEQ ID NO: 25) | |
| human BTG1 FW | CATCTCCAAGTTTCTCCGCACC |
| (SEQ ID NO: 26) | |
| human BTG1 RV | GCGAATACAACGGTAACCCGATC. |

Western blotting. Total cell lysates were extracted in lysis buffer (50 mM Tris-Cl (pH 7.5), 1 mM EDTA, 150 mM NaCl, 1%NP-40, 1% sodium deoxycholate, 1 mM PMSF, and protease inhibitor (Roche)). Anti-Hlx polyclonal rabbit antibody (SantaCruz, clone H-130, sc-135014) and anti-actin polyclonal goat antibody (Santa Cruz, clone C-11, sc1615) were used as primary antibodies, HPRT-conjugated anti-rabbit or anti-goat antibody (Santa Cruz) were used as secondary antibodies. ECL solution (Pierce) was used for detection.

Cell proliferation, cell cycle, and apoptosis assays. For cell proliferation assays, manual cell counts were performed by culturing cells in 24- or 48-well plates. Viable cells were counted using trypan blue exclusion and cell density was re-adjusted in each well every 3-5 days. For cell cycle assays, the Click-iT™ EdU Flow Cytometry Assay system (Invitrogen) was used following the manufacturer's instructions. For apoptosis assays, apoptotic and necrotic cells were analyzed by use of Annexin V/DAPI staining.

Colony, formation assays and serial replating assays. Assays were performed in MethoCult M3434 (Stern Cell Technologies, Vancouver, BC) containing IL-3, IL-6, SCF, and EPO or in MethoCult M3234 supplemented with M-CSF or GM-CSF as previously described (Higuchi et al, 2002; Huntly et al, 2004;Will et al, 2009). GFP+ colonies were scored 8-10 days after plating using an AXIOVERT 200M microscope (Zeiss, Maple Grove, Minn.). After scoring, GFP+ cells were re-sorted after each round and then serial replating assays were carried out. Cells were replated and colonies were again scored after 10-14 days.

Transplantation assays. For HLX overexpression studies, 5×104 lentivirus-transduced LinKit+ cells (Ly5.2) together with 2.5×105 spleen cells from congenic WT recipients (Ly5.1) were transplanted into lethally irradiated age-matched congenic WT recipients (Ly5.1) by retroorbital vein injection. Total body irradiation was delivered in a single dose of 950 cGy using a Shepherd 6810 sealed-source 137Cs irradiator.

Micorarray experiments and analysis. RNA was extracted from sorted GFP+ cells utilizing the RNeasy Micro Kit (Qiagen). After evaluation of the quality of RNA with an Agilent2100 Bioanalyzer, total RNA was used for amplification utilizing the Nugen Ovation pico WTA system according to the manufacturer's instructions. After labeling with the GeneChip WT terminal labeling kit (Affymetrix), labeled cRNA of each individual sample was hybridized to .Affymetrix Mouse Gene 1.0ST microarrays (Affymetrix), stained, and scanned by GeneChip Scanner 3000 7G system (Affymetrix) according to standard protocols.

Raw data were nointalized with the RMA algorithm of Affymetrix Power Tools v. 1.178. A t-test with Welch approximation for unequal group variances with p-values based on t-distribution was performed with a cutoff of p<0.05 (Hlx knockdown experiment) or p<0.1 (Mx overexpression experiment) in Multiple Experiment Viewer v,4 pilot2 (Saeed et al, 2006). Subsequently, probes with −log10(p) value <0.05 (or <0.1 for overexpression experiments) and a group mean difference >0.5 (log2 scale) were considered differentially expressed and used for further analysis. After filtering out unannotated and duplicate genes, the remaining genes were clustered by hierarchical clustering, using Euclidean distance, complete linkage clustering. For enrichment map analysis, gene enrichment table files were generated using the DAVID bioinformatics tool (Huang et al. 2009a; 2009b), filtered for significance with p-value and FDR thresholds set at <0.05 and <0,25, respectively, and visualized them using the Enrichment Map Cytoscape plugin (Cline et al, 2007; Merico et al, 2010). The gene lists were also analyzed by Gene Set Enrichment Analysis v2.0 (GSEA) (Mootha et al, 2003; Subramanian et al, 2005), using gene set size filters of min=8, max=500, the permutation type set to gene_set, MSigDB v3.0 gene sets (c2.cgp.v3.0.symbols.gmt) and a cutoff at p<0.05.

Data sets and statistical analyses. Analysis was carried out using the publicly available gene expression data sets with accession numbers GSE12417 (training set U1333A and U133B; test set U133plus2.0), GSE14468, and GSE10358 (ncbi.nlm.nih.gov/geo/). Clinical outcome and mutational data for the GSE10358 dataset were obtained from a recent study of the same group (Ley et al, 2010). Analyses of the gene expression profiles from GSE14468, GSE12417 training set and GSE10358 were performed based on published (Gentles et al, 2010) and publicly available MASS files (GSE24006) with reanalyzed data. For analysis of the test set of the GSE12417 dataset, CEL files were processed using GenePattern (Broad Institute, Cambridge Mass.) for normalization (ExpressionFileCreator algorithm). GraphPad Prism 5.0, R/Bioconductor sva package (R package version 3.2.0)(Leek et al., 2012), and SPSS 18.0 statistical package were utilized for further analyses.

All datasets were analyzed separately to dichotomize the population of patients of each dataset into subsets with high versus low expression of HLX transcript, using the 25th percentile of normalized HLX expression in each data set as the cutoff point. Publicly available clinical annotations accompanying each of these data sets were then used to perform Kaplan-Meier survival analysis (GraphPad Prism 5.0) comparing clinical outcome of patients with high versus low HLX expression. For PAK1 expression analysis, dichotomization at the 25th percentile and survival analysis was performed analogous to the study of HLX expression. To study the correlation between expression of PAK1 and HLX, expression datasets were RMA-normalized together and removal of batch effects was performed in R/Bioconductor using the sva package (R package version 3.2.0) (Leek et al., 2012). Multivariate analyses using Cox regression models were performed (with Forward Conditional and Backward Conditional methods in the SPSS 18.0 statistical package) using the cytogenetic risk data and mutational status information available for patients from the GSE 14468 dataset (including age (< or >60 years), gender, cytogenetic risk group, mutational status for FLT3ITD, FLT3D835 (TKD), NPM1, CEBPA, IDH1, IDH2, N-RAS, K-RAS, EVII expression and HLX expression status (low/high)). Confirmatory multivariate analysis was performed in the data of the GSE10358 dataset for the clinical and molecular parameters available for that dataset, including bone marrow blast percentage at diagnosis.

Signature generation and calculation of signature scores. CEL files for publicly available gene expression datasets GSE12417 U133A, GSE12417 U133plus2, GSE10358 and GSE14468 were downloaded from GEO and processed separately for each dataset in dChip (biosunl.harvard.edu/complab/dchip/) for generation of DCP files. Data were then normalized and modeled according to the preset normalization parameters of the software (probe selection method: invariant set; smoothing method: running median). Patients in each dataset were characterized as having low or high HLX levels, using the 25th percentile of normalized signal for the HLX probe in each dataset as the dichotomization point. Genes differentially expressed in patients with low versus high HLX were identified according to the following criteria: ratio of average expression of >1.2 or <1.2; absolute difference in average signal of >100; p-value<0.05; permutation testing (100 times) to assess false discovery rate (FDR) in each dataset. The 90th percentile of the number of probes with false discovery was used as a cutoff to exclude from further analysis the probes with the highest p-value among those that satisfied the other comparison criteria. For the generation of the HLX signature, the RMA-normalized log2-transformed GSE14468 gene expression data were dichotomized into HLX low and HLX high, as previously done for the survival analysis, and performed SAM analysis to identify differentially expressed genes between these groups. This list of genes was subsequently intersected with the human orthologs of the genes that were differentially expressed in the mouse overexpression or knockdown models, and which showed the same directionality of expression differences relative to Hlx levels between mouse and human data. This list of 45 genes was subsequently used as covariates for Hlx expression and for overall survival in the R/Bioconductor globaltest function (Goeman et al, 2004), and the most significantly correlated genes were selected to define an Hlx-associated signature (“HLX signature”). To calculate a signature score, expression of each aene was median-centered, and the mean of the positively associated minus the mean of the negatively associated genes was calculated for each patient sample. The samples from GSE14468 (test set) and GSE10358 (validation set) were ranked and dichotomized according to this normalized signature score (“HLX signature high” and “HLX signature low”).

Accession number. Complete array data are available in the Gene Expression Omnibus (GEO) of NCBI (Edgar et al., 2002), accession number GSE27947.

Short hairpin sequences. Short hairpin (sh) nucleic acid sequences used were:

luciferase target-
(SEQ ID NO: 27)
gtgcgttgttagtactaatcctattt
mouse Mx target-
(SEQ ID NO: 28)
ggcgcagaaggacaaggacaaggaagcgg
Non-silencing control-
(SEQ ID NO: 29)
acctccaccctcactctgccat
human HLX target-
(SEQ ID NO: 30)
ccctatgctgtgctcacga
human PAK1 #1-
(SEQ ID NO: 31)
CGATGAGAAATACCAGCACTA
human PAK1 #2-
(SEQ ID NO: 32)
GCGATCCTAAGAAGAAATATA.

These are DNA sequences which can be expressed by lentiviruses; cells are infected with these viruses, and then the cells transcribe the viral DNA into the actual shRNAs.

### Results

HLX overexpression impairs hematopoietic reconstitution and leads to a decrease in long-term hematopoietic stem cells and persistence of a small progenitor population. To examine the functional consequences of elevated HLX levels on hematopoiesis, lineage-negative (Lin-), Kit+ bone marrow (BM) cells were sorted from Ly5.2(CD45.2)±WT mice, transduced with a lentivirus expressing HLX and GFP, or GFP alone as a control, and transplanted into lethally irradiated congenic Ly5.1(CD45.1)+ recipient mice. Transduction efficiency of control lentivirus and 1711x lentivirus was comparable, with both at approximately 50% Twenty-four hours post-transplantation, both control and HLX-overexpressing GFP+ Ly5.2+ donor cells were detected in the BM at similar frequencies (42.8% and 41.6%, respectively), indicating equal homing of the transplanted cells. Twelve weeks after transplantation, hematopoietic multilineage reconstitution in the peripheral blood was evaluated. Both groups engrafted robustly with an average donor chimerism of Ly5.2 cells of 80% (SD: 10%) and 85% (SD: 9%) in the control and Hlx groups, respectively. However, while mice transplanted with control cells showed 35% (SD: 17%) GFP+ cells in the peripheral blood 12 weeks after transplantation, mice transplanted with Hlx-transduced cells displayed drastically less GFP+ cells with only 0.07% (SD: 0.06%), demonstrating a severe defect of HLX-overexpressing cells in hematopoietic reconstitution. To determine the cellular compartments in which HLX was effective, stem and progenitor cells in recipient BM were analyzed. No GFP+ long-term hematopoietic stem cells (LT-HSC; Thy1^{lo}Flk2-LSK (Lin-Scal+Kit+)) in mice transplanted with HLX-expressing cells were detected, while control mice displayed on average 42% (SD: 20%) GFP+ LT-HSC. Furthei more, in contrast to control animals, no GFP+ HLX-expressing short-term HSC (ST-HSCs; Thy1^{lo}Flk2+LSK), multipotent progenitors (MPP; Thy1-Flk2+LSK), common myeloid progenitors (CMP; Lin-Kit+Sca-1-FcγRII/IIIloCD34lo), granulocyte/monocyte progenitors (GMP; Lin-Kit+Sca-1-FcγRII/III+CD34+) or megakaryocyte/erythrocyte progenitors (MEP; Lin-Kit+Sca-1-FcγRII/III−CD34−) were found, indicating that HLX acts at the level of the earliest hematopoietic stem cells. When Hlx-GFP-transduced Lin-Kit+ (KL) cells were analyzed by AnnexinV/DAPI staining, both control and HLX-overexpressing cells displayed the same low percentage of apoptotic/necrotic cells, indicating that HLX acts by a mechanism other than induction of apoptosis or necrosis. Further analysis for donor-derived cell populations persisting upon HLX overexpression revealed a small population of GFP+, CD45,2(Ly,5.2)+, Lin−, CD34−, and Kit− cells which were still present in the BM 12 weeks after transplantation. Analysis for additional surface markers revealed that these cells were characterized by intermediate expression of CD11b, as well as high expression of CD49b and CD44, which is consistent with the surface phenotype of myeloid precursor cells slightly past the GMP stage (Novershtern et al, 2011). This CD45+CD11bmidCD49b+CD44+ cell population was 16-fold expanded upon HLX expression in comparison to control (p=1.1×10-5).

HLX confers unlimited serial clonogenicity to CD34-Kit-hematopoietic cells. Next, in vitro colony formation assays of transduced LSK cells were performed. Hlx-transduced LSK cells formed slightly fewer and smaller colonies than control-transduced LSK cells in the initial plating. To evaluate long-term clonogenic potential of HLX-overexpressing cells, serial-replating assays were perfoiiiied. LSK cells overexpressing HLX showed greater clonogenic capacity in the 2nd to 5th plating in comparison to control-transduced cells, and maintained serial replating capacity through the 6th to 9th plating, showing a de facto immortalization of this clonogenic progenitor population by HLX. In addition, colonies were noticeably larger in size after five platings compared to control. Analysis of cells isolated from the initial plating revealed that HLX overexpression led to a decrease of Kit+ cells, similar to the in vivo phenotype, and an increased proportion of phenotypically more mature CD34-Kit− cells in comparison to control-transduced cells. To further characterize this persisting population, a panel of cell surface markers was examined. While the CD34-Kit− cells were negative for CD11 c, CD25, FcγRII/III, CD61, CD115, and CD 150, they expressed CD49b and CD44, as well as intermediate levels of CD11b, similar to the observations in vivo. To determine which cellular subpopulation conferred the increased clonogenic capacity, equal numbers of CD34+Kit+ cells, CD34+Kit− cells, CD34-Kit+ cells, and CD34-Kit− cells were sorted from the first plating (populations I-IV), and subjected each individual population to colony formation assays. Only CD34-Kit− cells derived from HLX-overexpressing cells formed a larger number of colonies in comparison to control cells, while all other populations did not display significant clonogenicity. Furthermore, the HLX-overexpressing GFP4-CD34-Kit− cells showed serial replating capacity through 4 rounds, while all other populations exhausted significantly earlier. Finally, when HLX-overexpressing GFP+CD34-Kit− cells from the fourth, sixth, or eighth plating were injected into irradiated NOD-SCID-IL2Rgamma null (NSG) mice, GFP+ cells were detectable after 7 weeks in the peripheral blood. These data indicate that increased levels of HLX confer long-term clonogenicity to a population of CD34-Kit− cells.

HLX induces a myelomonocytic differentiation block. To investigate the effect of HLX overexpression on differentiation capacity, the clonogenic GFP+CD34-Kit− cells from the first colony assay were analyzed for the expression of additional cell surface markers. The proportions of Gr1+Mac1+ and Gr1-Mac1+, as well as F4/80+Mac1+ expressing cells were significantly reduced (Gr1+Mac1+: from 29.7% (control) (SD: 6.6%) to 16.2% (Hlx) (SD: 1.9%), p=0.026; Gr1-Mac1+: from 30.7% (SD: 4.5%) to 11.2% (SD: 2.6%), p=0.003; F4/80+Mac1+: from 31.6% (SD: 4.9%) to 8.9% (SD: 2.8%), p=0.002; N=3), indicative of a defect in myelomonocytic differentiation. Expression of erythroid, B-lymphoid, and T-lymphoid markers was unchanged. Furthermore, almost half of the HLX-overexpressing GFP+CD34-Kit− population (47.6%, (SD: 2.8%)) was lineage-negative, in contrast to only 17% (SD: 1.2%) of GFP+CD34-Kit− control-transduced cells (p=0.005). Sorted, HLX-overexpressing GFP±Lin-CD34-Kit− cells also showed a significant increase in clonogenicity compared with control-transduced cells, indicating that HLX acts at the level of Lin-CD34-Kit− cells. To specifically test myelomonocytic differentiation, colony-formation assays were carried out with GM-CSF or M-CSF stimulation. Hlx-transduced cells gave rise to lower numbers of Gr1-Mac1+ and F4/804-Mac1+ cells compared to control-transduced cells, upon either GM-CSF or M-CSF stimulation. Cytomorphological evaluation of cells after stimulation showed an increased percentage of Hlx-transduced cells with immature progenitor morphology, in stark contrast to control-transduced cells which predominantly displayed mature monocytic morphology. Taken together, the findings show that HLX not only enhances clonogenicity of an increased population of Lin-CD34-Kit− cells, but also confers a partial myelomonocytic differentiation block.

HLX downreguiation inhibits AML. To test whether HLX overexpression is functionally important for AML, Hlx was targeted by RNA interference. Leukemia cells derived from the PU.1 URE^{Δ/Δ} AML model (URE cells) as well as human AML cells were transduced with lentiviral constructs expressing either Hlx-directed (sh Hlx) or control shRNA (sh control), Knockdown of HLX by 80% led to significantly reduced cell proliferation in suspension culture, as well as reduced formation of colonies of URE cells in methylcellulose assays in comparison to control cells (p<0.00001). Likewise, human KG1a, THP1, and MOLM13 AML cells showed 40-60% reduced growth (p=0.003, p=0.001, and p=0.014, respectively) in suspension culture, as well as significantly diminished clonogenicity (p=0.02, p=0,038, and p=0.004, respectively). To test the anti-leukemic effect of HLX suppression in vivo, murine transplantation assays were performed. Reduction of HLX levels in transplanted URE cells significantly prolonged recipient survival in comparison to mice transplanted with control snRNA-transduced cells (p=0.0012, and p=0.0041). Taken together, the findings demonstrate that HLX is functionally important for AML cells, and that targeting HLX can inhibit cellular growth, decrease clonogenicity, and lead to improved survival in a murine AML transplantation model. Of note, Hlx heterozygous mice, which show a 50% reduction of HLX protein levels in BM cells, did not show any noticeable effects on normal hematopoiesis, including hematopoietic stem cell functions such as capacity for hematopoietic reconstitution and serial transplantability.

Inhibition of HLX leads to decreased cell cycling, increased cell death, and differentiation of AML cells, and causes significant changes in gene expression. To obtain insight into the mechanism of action of HLX inhibition, the cell biological and molecular consequences of knockdown of HLX were studied in leukemia cells. HLX downregulation in URE cells led to a decrease in viable cells, and an increase in necrotic cells. This was accompanied by a lower number of cells in S phase (p=0.027), and a higher number of cells in G0/G1 phase of cell cycle (p=0.002). In addition, reduction of HLX led to an increased population of cells expressing lower levels of Kit and higher levels of Gr-1 and Mac1, indicative of myeloid differentiation. Stimulation with GM-CSF further increased the number of Mac1 and Gr-1 expressing cells, and led to increased differentiation of AML cells upon sh Hlx treatment in comparison to control-treated cells, which retained an immature, leukemic morphology. This observation shows that inhibition of HLX can overcome the myeloid differentiation block of AML cells.

To gain insight into the molecular effects caused by HLX inhibition, gene expression profiles were measured for sh Hlx-transduced URE cells and control shRNA-transduced cells. Leukemia cells treated with Mx-directed shRNAs displayed an altered gene expression pattern with 392 genes being significantly differentially expressed (mean difference>0.5 (log2), p<0.05). Gene set enrichment analysis (GSEA) showed that “leukocyte differentiation”, “cell activation”, and “cell proliferation” were among the most significantly affected cellular functions, which is consistent with the leukemia-inhibitory effect of HLX reduction in LIRE cells. Several key genes involved in the regulation of cell cycle and proliferation, cell death, and myeloid differentiation, were significantly changed upon HLX downregulation. Differential expression of several genes, namely Btg1, FoxO4, Fyn, Gadd45a, RhoB, Trp63, Zfp3611, 1Hdac7, and Pak1 by qRT-PCR was confirmed. Enrichment of genes involved in pathways of other cellular functions was also observed. Further, GSEA was utilized to compare the Hlx knockdown data with the molecular signatures database (MSigDB) (Mootha et al, 2003; Subramanian et al, 2005); negative enrichment of several known leukemia- and stem cell-related gene signatures was found. Taken together, these data are consistent with a model that HLX overexpression leads to activation of a specific transcriptional program in leukemia cells which affects processes critical for leakemogenesis such as cell differentiation and proliferation, and which can at least partially be reversed by inhibition of HLX.

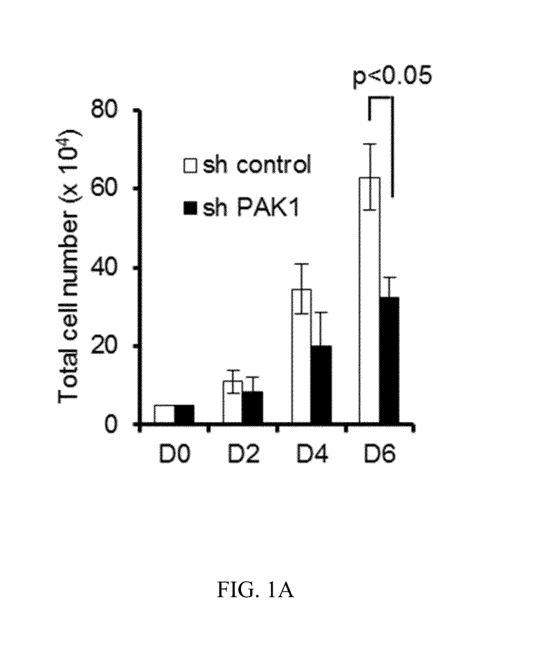

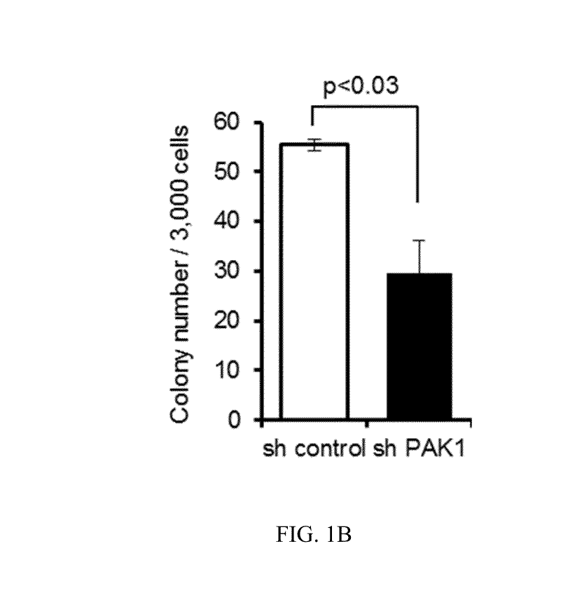

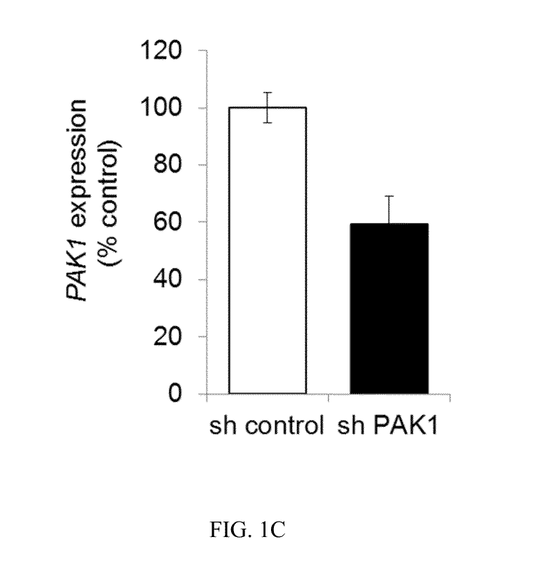

Focus was directed to two downstream genes modulated upon HLX inhibition in AML cells, Pak1 and Btg1, which have previously been implicated in the regulation of cell cycle and malignant proliferation (Ong et al, 2011; Kuo et al, 2003). Knockdown of PAK1 in KG1a AML cells led to a significant inhibition of cell proliferation (p<0.05) and clonogenicity (p<0.03) (FIG. 1), mimicking the effects observed by knockdown of HLX. Similarly, shRNA knockdown of PAK1 reduces cell proliferation and colony formation in THP1 AML cells (not illustrated). shRNA knockdown of PAK1 also induces apoptosis and differentiation of THP1 cells (not illustrated). Ectopic expression of BTG1 led to a significant inhibition of cell growth (p<0.01) and colony forming capacity (p<0.008) of KG1a cells. These findings indicate that downregulation of PAK1 and upregulation of BTG1 are functionally relevant for mediating the leukemia-inhibitory effects of HLX knockdown.

HLX is overexpressed in patients with AML. To examine whether HLX overexpression plays a role in human leukemia, gene expression data were analyzed from 354 patients with AML (Figueroa et al, 2010; Wouters et al, 2009). HLX was overexpressed in 87% of patients with AML in comparison to CD34+ cells of healthy donors. On average, HLX expression was 2.03-fold higher in AML patients (p=1.9×10-9). 54% (190 out of 354) of patients with AML overexpressed HLX more than 2-fold, with the range extending up to 6.8-fold overexpression. These results demonstrate that HLX overexpression is a common feature in patients with AML. HLX overexpression was found across different French-American-British (FAB) classification subsets of AML. Of note, high HLX expression was significantly more frequent in AML with myelomonocytic (M4) and monoblastic (M5) morphology, which is consistent with the observed myelomonocytic differentiation block caused by HLX overexpression in vitro.

Increased HLX expression correlates with inferior survival. It was examined if HLX expression levels in patients were associated with known clinical or molecular parameters. Four published datasets were analyzed for patients with AML, of whom gene expression and time-to-event data were available (NCBI GEO accessions GSE10358, GSE12417 (U133A), GSE 12417 (U133plus2), and GSE 14468 (Metzeier et al, 2008; Tomasson et al, 2008; Wouters et al, 2009)). As the lower 25% of patients had HLX expression levels very similar to CD34+ cells of healthy donors, the 25th percentile was used to dichotomize patients into “HLX high” and “HLX low” expressers. The overall survival of AML patients was compared with low versus high HLX. In each of the 4 different data sets, high HLX expression was associated with inferior overall survival. Overall survival (irrespective of HLX status) in datasets GSE12417 (U133plus2.0), GSE14468 and GSE10358 was very similar, with superimposable survival curves (p=0.4636, log-rank test), suggesting that these cohorts and their clinical outcomes could be combined for further analyses. The GSE12417 (U133A) dataset showed a worse overall survival outcome compared with the other datasets and was therefore not included in the combined analysis. Consistent with the analyses of the individual datasets, the evaluation of the combined set of patients from the GSE 10358, GSE 12417 (U133plus2.0) and GSE14468 datasets (N=601 total) confirmed that high HLX levels are associated with inferior overall survival (p=2.336×10-6 (log-rank); hazard ratio (HR)=0.57 (95% CI: 0.046-0.71); median survival: 17.05 months (HLX high), “not reached” (HLX low); 5-yr survival rate: 32.95% (HLX high), 55.85% (HLX low)). To assess whether the impact of HLX expression on overall survival is independent of known prognostic factors for AML, a multivariate analysis was performed based on the data of the GSE14468 dataset (Figueroa et al, 2010; Wouters et al, 2009), using a Cox regression model. In this analysis, high HLX status remained an independent prognostic factor (p=0.0416, HR 1.521) along with FLT3 mutation status (p=0.0003, HR 1.925), NPM I mutation status (p=0.0006, HR 0.518), CEBPA mutation status (p=0.0371, HR 0.693), and cytogenetic risk group (p=0,0109, HR 1.382). Of note, the relative blast percentage in the bone marrow was not correlated with HLX expression or overall survival. The independent prognostic role of HLX status indicated that it may provide additional prognostic information for patients who belong to previously established, molecularly defined subtypes of AML. Among patients with FLT3 WT, NPM1 mutations, or CEBPA mutations, high HLX expression was associated with inferior overall survival (p=0.0175, p=0,0407 and p=0.0306, respectively).

An HLX-dependent transcriptional signature is functionally relevant in AML patients. To obtain insight into the molecular consequences of elevated HLX levels, HLX was overexpressed in sorted murine LSK cells, and a genome-wide transcriptional analysis was performed. 195 genes were significantly changed, resulting in a clearly distinguishable expression signature induced by HLX overexpression. Gene set enrichment analysis revealed enrichment of known leukemia- and stem cell-related gene signatures, consistent with previous findings.

Next, it was tested whether this mouse LSK HLX overexpression gene set correlated with HLX expression in human AML patients. Specifically, the human orthologs of the mouse gene set were compared to HLX expression levels of AML patients in the different cohorts using globaltest (Goeman et al, 2004). A highly significant correlation was found between the mouse gene signature and HLX expression in human AML samples (p=7.43×10-23 for GSEI4468, p=2.13×10-08 for GSE10358, p=2.31×10-06 for GSE12417 (U1133plus2.0), and p=5.01×10-10 for GSE12417 (U133A)). Further, differentially expressed genes from the HLX overexpression or inhibition studies were intersected with analogously differentially expressed genes in “HLX high” versus “HLX low” patients of the GSE14468 data set, and analyzed for association with survival, Thereby, a HLX-dependent core set of 17 genes (referred to as “HLX signature”) was defined correlating with HLX expression status in patients with AML. When patients were dichotomized into “HLX signature high” versus “HLX signature low” patients (defined by the genes of the signature, excluding HLX), “HLX signature high” patients had significantly inferior overall survival (p=0.0089 (log-rank); hazard ratio (HR)=0.66 (95% CI: 0.48 to 0.90); median survival: 17.22 months for HLX signature high, “not reached” for HLX signature low; 5-yr survival rate: 34.5% for HLX signature high, 53.9% for HLX signature low).

The HLX signature was subsequently validated in an independent cohort of patients, the GSE10358 data set; the signature correlated strongly (p=7.8×10-11) with “HLX high” versus “HLX low” expression status in these AML patients as well. Furthermore, “HLX signature high” patients showed a strikingly inferior overall survival (p=1.89×10-05 (log-rank); hazard ratio (HR)=0.42 (95% CI: 0.28 to 0.62); median survival: 18.3 months (HLX signature high), “not reached” (HLX signature low); 5-yr survival rate: 29.0% (HLX signature high), 67.0% (HLX signature low)). Taken together, these data suggest that elevated HLX levels cause a specific, functionally critical gene expression signature in human AML.

Interestingly, PAK1 was part of the HLX-induced prognostic signature. Given the finding that PAK1 mediates the leukemia-inhibitory effects of HLX knockdown in AML cells, it was asked whether PAK1 expression levels alone may be functionally relevant in AML patients. AML patients were dichotomized into “PAK1 high” and “PAK1 low” expressers and clinical outcome was analyzed. “PAK1 high” patients showed significantly inferior overall survival (p=0.00014 (log-rank)) than “PAK1 low” patients (median: 17.7 months (PAK1 high) vs. 109.1 months (PAK1 low); 5-yr survival rate: 34.0% (PAK1 high) vs. 50,5% (PAK1 low)). Of note, high PAK1 expression was associated with inferior overall survival only in patients of the “HLX high” group (p=0.0005 (log-rank); hazard ratio (HR)=0.62 (95% CI: 0.48-0.81)); median survival: 15.8 months (PAK1 high), 42.0 months (PAK1 low); 5-yr survival rate: 29,7% (PAK1 high), 48.1% (PAK1 low)), but not in “HD(low” patients (p=0.77 (log-rank); hazard ratio (HR)=1.08 (95% CI: 0.65-1.78)); 5-yr survival rate: 55.0% (PAK1 high), 55.0% (PAK1 low)). In addition, PAK1 expression levels were on average 1.5-fold higher (p=2.2×10-16) in “HLX high” patients compared to “HLX low” patients, and HLX and PAK1 expression levels in individual patients were also significantly correlated (p=8.8×10-15, R=0.31). Furthermore, experimental overexpression of HLX in LSK cells led to a significant increase in Pak1 mRNA expression (1.9-fold, p=0.017).

### Discussion

Utilizing both mouse and human model systems, the class II homeobox protein HLX was shown to affect hematopoietic stem cell function, as well as clonogenicity and differentiation of immature hematopoietic progenitor cells. HLX is significantly overexpressed in the majority of patients with AML, and high HLX expression levels are independently associated with inferior clinical outcome. Thereby, the present study identifies HLX as a class II homeobox gene that is critically involved in the pathogenesis of acute myeloid leukemia. The finding that increased HLX expression correlates with more aggressive disease, combined with the observation that HLX knockdown results in an inhibition of growth and clonogenicity of leukemia cells further indicate that HLX is a promising prognostic and therapeutic target.

Many clustered (class I, or HOX) homeobox genes have been implicated in normal hematopoiesis as well as leukemia, but much less is known about the role of non-clustered (class II) homeobox transcription factors (for review see (Argiropoulos and Humphries, 2007)). Several HOX genes are expressed at high levels in subtypes of AML (Alcalay et al, 2005; Ayton and Cleary, 2003; Bullinger et al, 2004; Horton et al, 2005). Important roles in leukemic transformation have been demonstrated specifically for several members of the HOX-A and the HOX-B cluster (Fischbach et al, 2005; Krivtsov et al, 2006; Kroon et al, 1998; Sauvageau et al, 1997; Somervaille and Cleary, 2006; Thorsteinsdottir et al, 1997). Also, the non-clustered homeobox gene CDX2 was recently reported to be implicated in leukemogenesis (Scholl et al, 2007). Expression levels of homeobox gene HLX are strongly associated with inferior overall survival in several large, independent cohorts of patients with AML. Furthermore, the prognostic value of HLX is a broad phenomenon across several molecular subsets of patients, and HLX holds up as an independent prognostic factor in a multivariate model. Gene expression analyses upon experimental overexpression and inhibition of HLX demonstrated that HLX regulates the expression of a specific subset of genes, and that part of this “HLX signature” is also detectable in AML patients and discriminates between patients with poor and favorable clinical outcome. Taken together, these observations suggest that HLX is a key reaulator of a gene subset critical for AML pathogenesis, and that it may define a previously unrecognized molecular subtype of AML with distinct biological features and clinical outcome.

PAK1 and BTG1 were identified as two genes downstream of FILX in myeloid cells, and PAK1 and BTG1 are functionally relevant in AML cells. Notably, PAK1 is upregulated in human AML patients with high HLX expression, and it is also part of the HLX-induced core signature that independently predicts overall survival in AML patients. Interestingly, high PAK1 expression itself is associated with inferior overall survival, but only in patients with high HLX expression, indicating that there is an interaction of HLX and PAK1 with respect to their impact on overall survival. The correlation of HLX and PAK1 expression levels in the murine HLX knockdown model and in patients with AML, together with the observed induction of Pak1 expression upon HLX overexpression in stem cells strongly suggest that Pak1 is a downstream target of HLX and critically mediates, at least in part, the leukemia-promoting effects of HLX in patients in vivo.

Several HOX genes, such as HOXB4, have been reported to be stimulators of HSC function and expansion (Antonchak et al, 2002; Sauvageau et al, 1995). The present data show that HLX suppresses the function of normal immature HSC and progenitors, however leads to an increase of clonogenicity, as well as a differentiation block at the level of phenotypically more mature progenitors. As the loss of HSC does not seem to be mediated by induction of apoptosis or necrosis. FILX may exert this dual role by triggering initial differentiation of HSC and suppression of terminal differentiation at a more committed progenitor level. While overexpression of HLX led to the formation of myeloid progenitors with unlimited serial clonogenicity, no development of leukemia upon transplantation was observed, suggesting that HLX elevation alone may not be sufficient for full transformation. Like other homeobox genes, Hlx may function in concert with co-factors (Moens and Selleri, 2006; Pineault et al, 2004). Such co-factors could confer cell type specificity to the effects of HLX overexpression, and also contribute to leukemic transfainiation. It is also possible that increase of HLX plays a role in leukemia maintenance rather than leukemia induction.

Several transcription factors that govern normal hematopoietic differentiation have been implicated in leukemogenesis by blocking differentiation and promoting self-renewal and clonogenicity (for review see (Rosenbauer and Tenen, 2007)). HLX may act similar to those factors by establishing a specific gene expression program in early HSPC, which results in increased long-term clonogenicity and a differentiation arrest, contributing to poor clinical outcome. Thus, HLX expression levels may be utilized to predict clinical outcome and improve risk stratification.

### Example II—PAK1 Inhibition Inhibits Growth of Acute Myeloid Leukemia and Myelodysplastic Syndrome Cells

### Summary

In Example 1, studies were presented showing shRNA mediated inhibition of PAK1 levels. These studies are extended in the present Example to demonstrate pharmacological inhibition of PAK1 activity and levels by different compounds. One compound, 1,1′-Disulfanediyldinaphthalen-2-ol, targets the autoregulatory mechanism and promotes the inactive conformation of PAK1. A second compound, (S)-N-(2-(dimethylamino)-1-phenylethyl)-6,6-dimethyl-3-((2-methylthieno[3,2-d]pyrimidin-4-yl) amino)-4,6-dihydropyrrolo[3,4-c]pyrazole-5(1H)-carboxamide, is an ATP-competitive inhibitor of the kinase domain.

### Results and Discussion

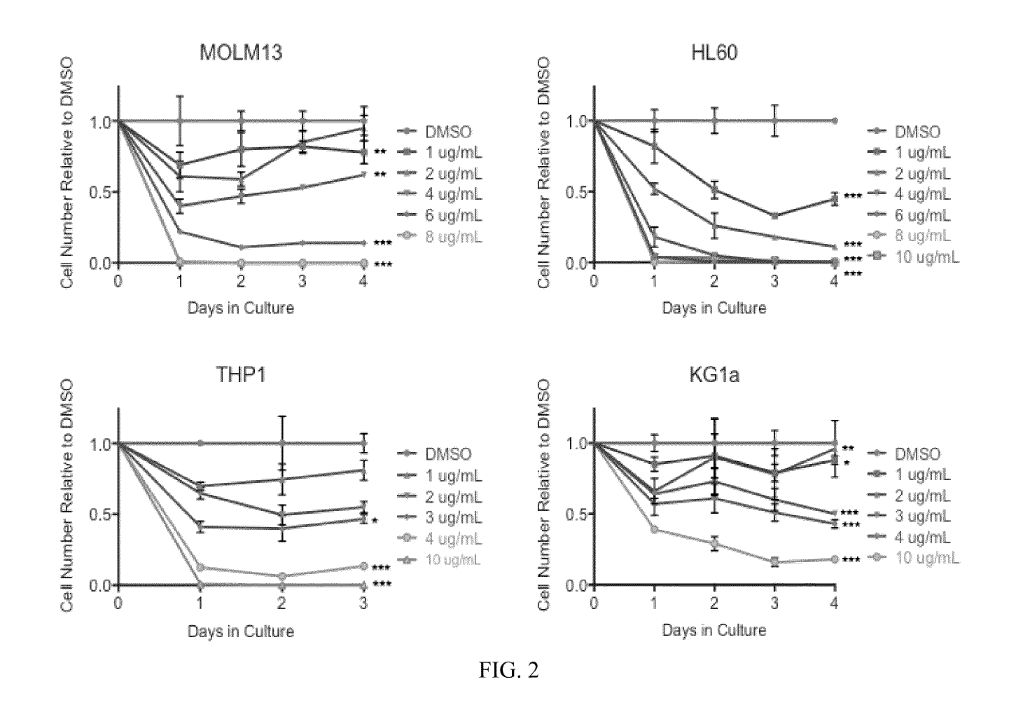

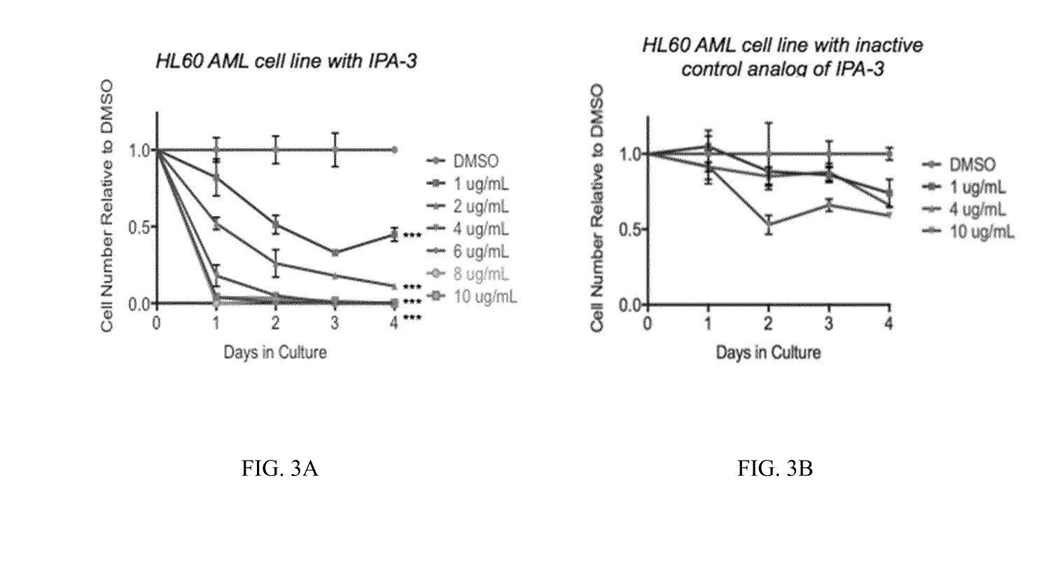

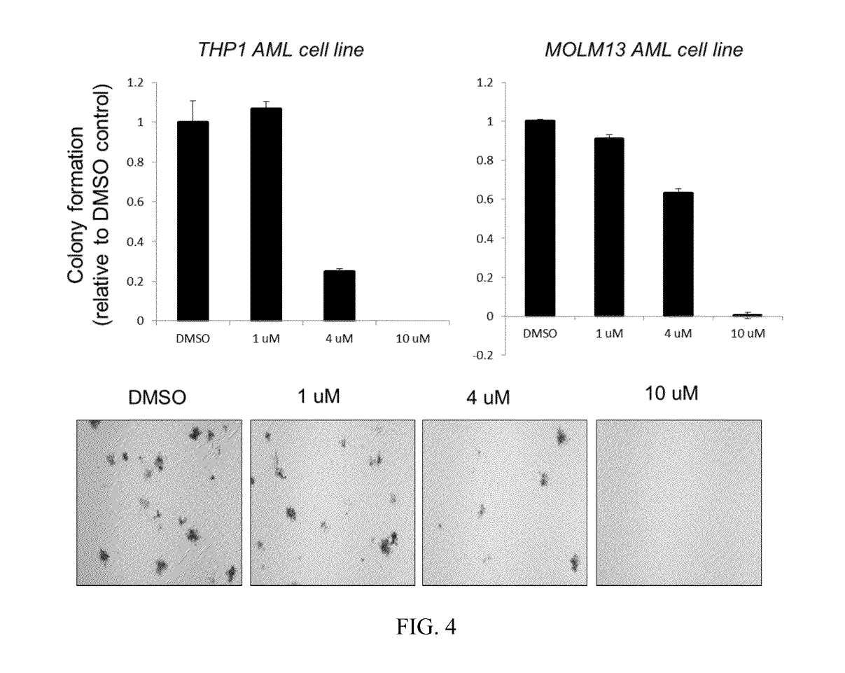

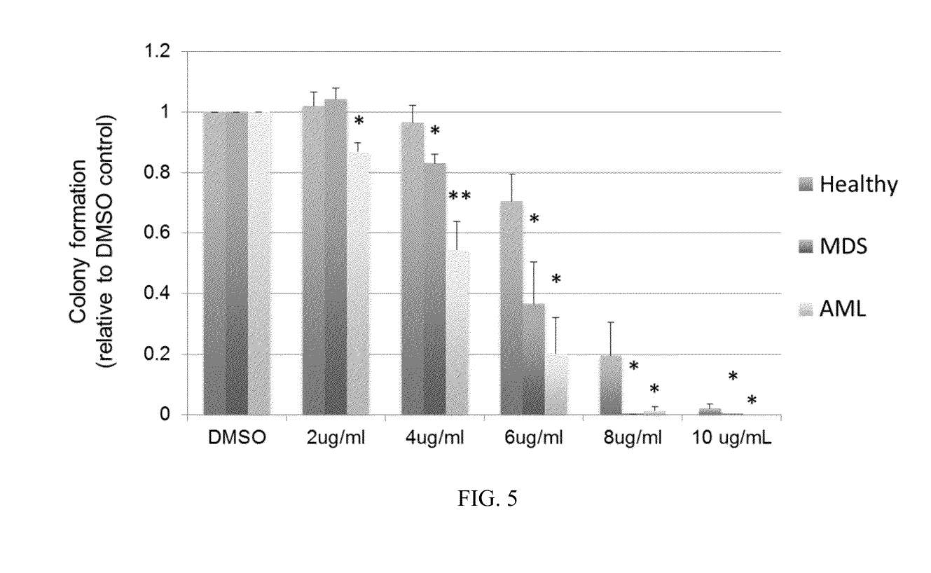

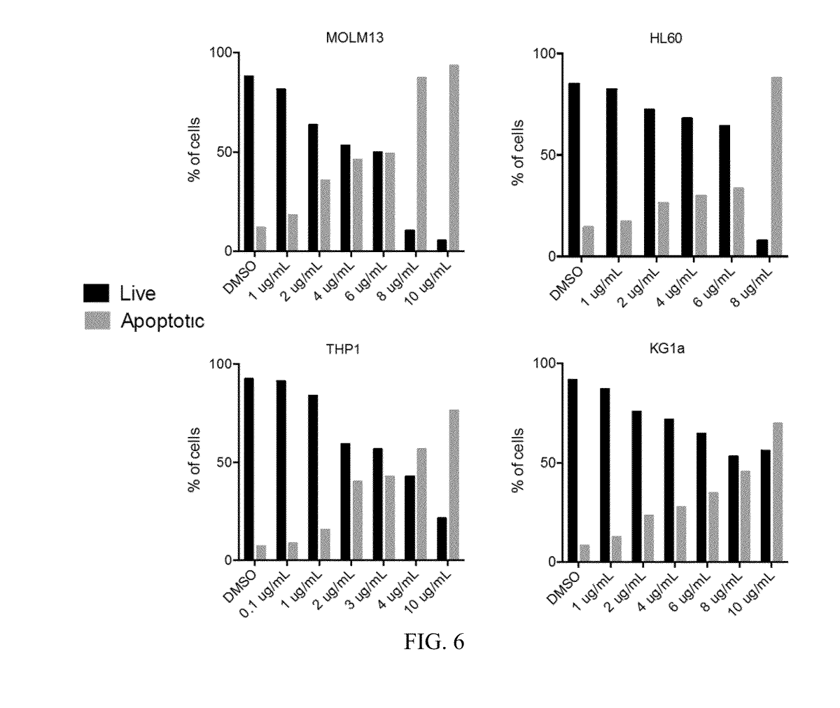

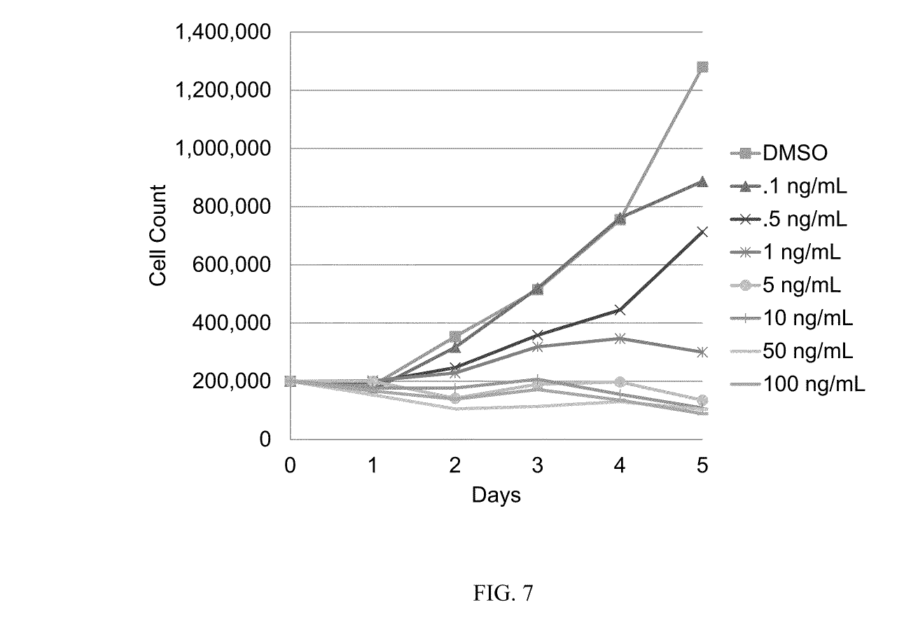

Compound 1,1′-Disulfanediydinaphthalen-2-ol (herein referred to as “Compound 1”) reduces proliferation of AML cells as demonstrated in four different AML cell lines (HL60, THP1, KG1a and MOLM13) (FIG. 2). In contrast, an inactive control analog of Compound 1 does not have the same effect on AML cells. FIG. 3A shows the effects of Compound 1 on reducing proliferation of HL60 AML cells. In contrast, an inactive control analog of Compound 1 is not effective in the same cell line (FIG. 3B). In addition, inhibition of PAK1 by Compound 1 reduces colony formation ability of AML cells (FIG. 4). As shown in FIG. 5, Compound 1-mediated inhibition of PAK1 has a greater effect on growth of AML cells than on healthy hematopoietic cells. The experiments illustrated in FIG. 5 were performed using primary bone marrow and peripheral blood samples given by healthy donors and by patients with acute myeloid leukemia. Inhibition of PAK1 by Compound 1 induces apoptosis in AML cells (FIG. 6). Inhibition of PAK1 by (S)-N-(2-(dimethylamino)-1-phenylethyl)-6,6-dimethyl-3-((2-methylthieno[3,2-d]pyrimidin-4-yl)amino)-4,6-dihydropyrrolo[3,4-c]pyrazole-5(1H)-carboxamide (“Compound 2”) also reduces proliferation of AML cells (FIG. 7).

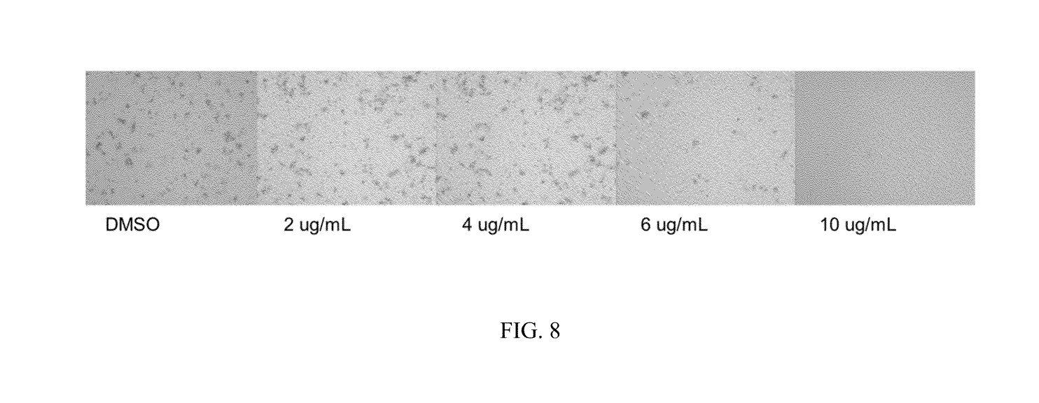

FIG. 8 illustrates Compound 1-mediated inhibition of PAK1 effects on colony formation of cells from a patient with myelodysplastic syndrome (MDS) Inhibition of PAK1 is effective in both myelodysplastic syndrome (MDS) and acute myeloid leukemia (AML). See also FIG. 5.

Different strategies have been illustrated herein to target PAK1 in myelodysplastic syndrome (MDS) and acute myeloid leukemia (AML) cells: sh-RNA mediated inhibition to reduce PAK1 expression, chemical inhibition (autoregulatory domain) by Compound 1, and chemical inhibition (ATP-competitive inhibition of kinase activity) by Compound 2. Correlation data with HLX expression indicates that taraeting of PAK1 is a therapeutic strategy in both AML and MDS, particularly in patients with high HLX expression. Targeting of PAK1 is also expected to be effective in other cancer types with high HLX levels.

### Example III—Effects of PAK1 Inhibition on Downstream Signaling Pathways

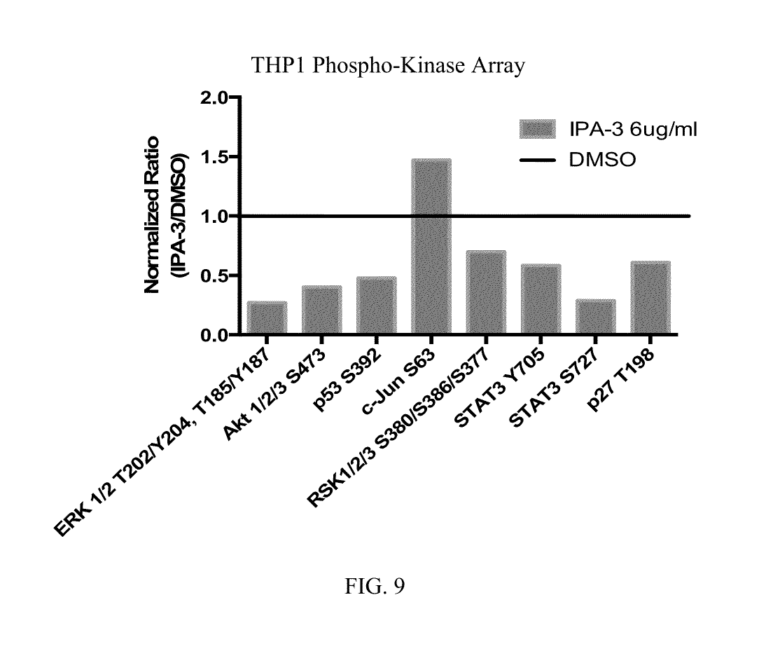

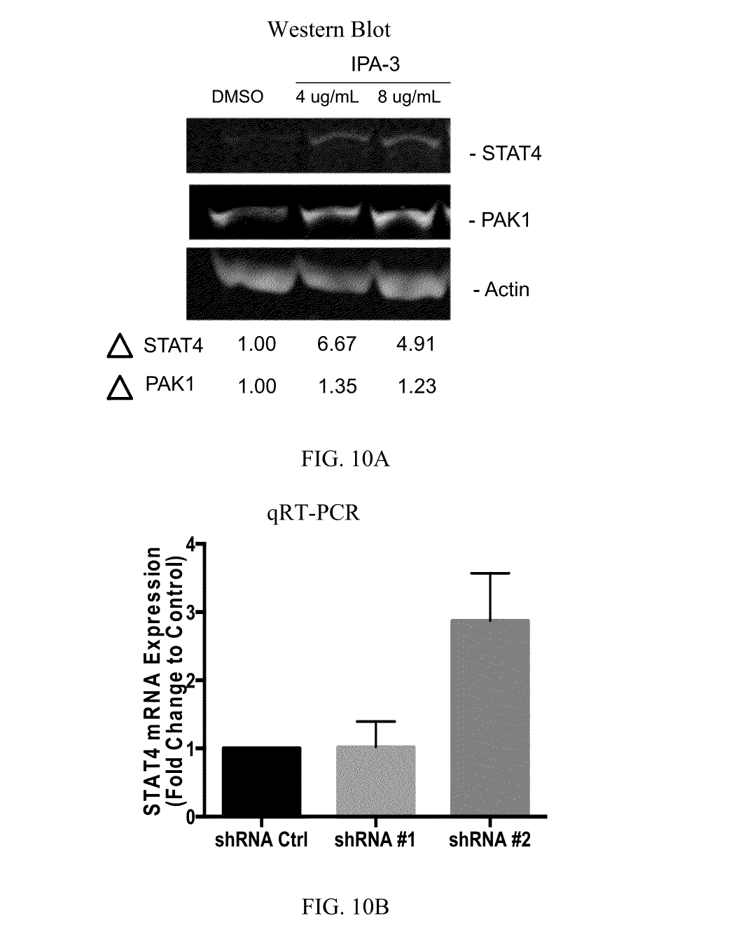

Measurement of phosphorylation status of kinases in THP1 leukemia cells after 4 hours of treatment with 6 μg/ml of IPA-3 or DMSO demonstrates on-target effects of Compound 1 (Akt 1/2/3 S473) and that several additional oncogenic signaling pathways are significantly inhibited in AML cells in response to PAK1 inhibition (FIG. 9). In contrast, STAT4 expression is increased upon treatment with Compound 1 or PAK1 shRNA knockdown in THP1 cells (FIG. 10).

### Example IV—Increased Survival With Lower PAK1 Levels

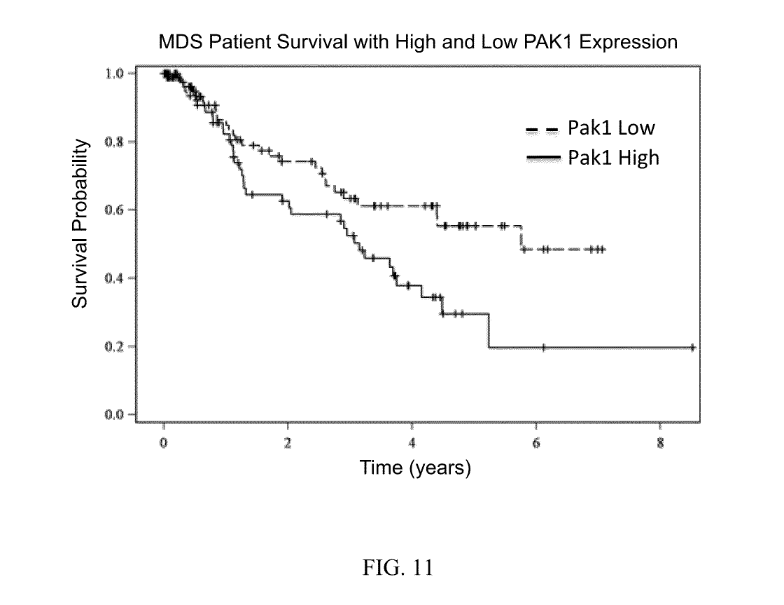

Patients with myelodysplastic syndromes (MDS) show significantly decreased overall survival when they express high PAK1 levels, in comparison to MDS patients with low PAK1 levels (FIG. 11). This finding shows that higher PAK1 levels are associated with more aggressive disease, suggesting PAK1 as a therapeutic and diagnostic/prognostic target in MDS.

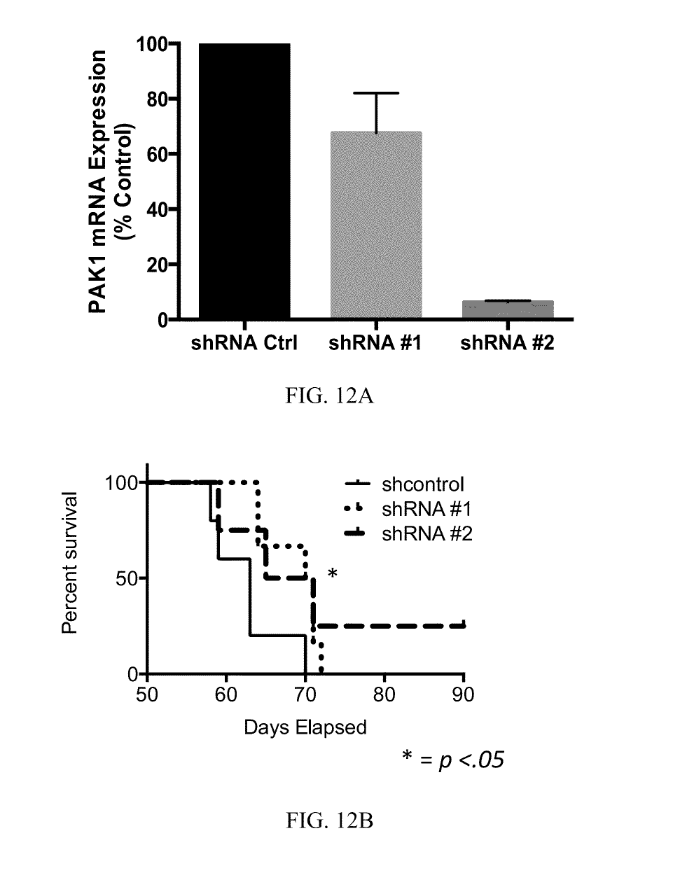

The effects on survival of reduced PAK1 expression in THP1 AML cells when expression was reduced by shRNAs was evaluated in mice. FIG. 12A shows reduction of PAK1 expression in THP1 AML cells by shRNAs. Significant prolongation of survival was observed in mice injected with 300,000 THP1 cells expressing either a shRNA #1 or shRNA #2 to PAK1 compared to THP expressing a non-silencing control shRNA (FIG. 12B). Of note, some mice treated with shRNA42 show long-term survival during the entire duration of the assay (90 days). Significantly less liver infiltration of leukemia cells was observed in animals receiving THP1 with shRNA #1 or #2 compared to control cells (not illustrated).

### REFERENCES

Alcalay, N I., Tiacci, E., Bergomas, R., Bigerna, B., Venturini, E., Minardi, S. P., Meani, N., Diverio, D., Bernard, L., Tizzoni, L. et al . (2005). Acute myeloid leukemia bearing cytoplasmic nucleophosmin (NPMc+ AML) shows a distinct gene expression profile characterized by up-regulation of genes involved in stem-cell maintenance. Blood 3, 899-902.
Allen, I D., Lints, T., Jenkins, N. A., Copeland, N. G., Strasser, A., Harvey, R. P. and Adams, J. M. (1991). Novel murine homeo box gene on chromosome 1 expressed in specific hematopoietic lineages and during embryogenesis. Genes Dev. 4, 509-520.
Antonchuk, J., Sauvageau, G. and Humphries, R. K. (2002). HOXB4-induced expansion of adult hematopoietic stem cells ex vivo. Cell 1, 39-45.
Argiropoulos, B. and Humphries, R. K. (2007). Hox genes in hematopoiesis and leukemogenesis. Oncogene 47, 6766-6776.
Ayton, P. M. and Cleary, M. L. (2003). Transformation of myeloid progenitors by MLL oncoproteins is dependent on Hoxa7 and Hoxa9. Genes Dev. 18, 2298-2307.
Bullinger, L., Dohner, K., Bair, E., Frohling, S., Schlenk, R. F., Tibshirani, R., Dohner, H. and Pollack, J. R. (2004). Use of gene-expression profiling to identify prognostic subclasses in adult acute myeloid leukemia. N. Engl. J. Med. 16, 1605-1616.
Cline, M. S., Smoot, M., Cerarni, E., Kuchinsky, A., Landys, N., Workman, C., Christmas, R., Avila-Campilo, I., Creech, M., Gross, B. et al . (2007). Integration of biological networks and gene expression data using Cytoscape. Nat. Protoc. 10, 2366-2382:
Deguchi, Y. and Kehrl, J. H. (1991). Selective expression of two homeobox genes in CD34-positive cells from human bone marrow. Blood 2, 323-328.
Deguchi, Y., Kirschenbaum, A. and Kehrl, J. H. (1992). A diverged homeobox gene is involved in the proliferation and lineage commitment of human hematopoietic progenitors and highly expressed in acute myelogenous leukemia. Blood 11, 2841-2848.
Edgar, R., Domrachev, M. and Lash, A. E. (2002). Gene Expression Omnibus: NCBI gene expression and hybridization array data repository. Nucleic Acids Res. 1, 207-210.
Figueroa, M. E., Lugthart, S., Li, Y., Erpelinck-Verschueren, C., Deng, X., Christos, P.J., Schifano, E., Booth, J., van Putten, W., Skrabanek, L, et al . (2010), DNA methylation signatures identify biologically distinct subtypes in acute myeloid leukemia. Cancer. Cell. 1, 13-27.
Fischbach, N. A., Rozenfeld, S., Shen, W., Fong, S., Chrobak, D., Ginzinger, D., Kogan, S. C., Radhakrishnan, A., Le Beau, M. M., Largman, C. and Lawrence, H. J. (2005). HOXB6 overexpression in murine bone marrow immortalizes a myelomonocytic precursor in vitro and causes hematopoietic stem cell expansion and acute myeloid leukemia in vivo. Blood 4, 1456-1466.
Gentles, S. K., Majeti, R. and Alizadeh, A. A. (2010). Association of a leukemic stem cell gene expression signature with clinical outcomes in acute myeloid leukemia. JAMA 24, 2706-2715.
Goeman, J. J., van de Geer, S. A., de Kort, F. and van Houwelingen, H. C. (2004). A global test for groups of genes: testing association with a clinical outcome. Bioinformatics 1, 93-99.
Hanahan D, Weinberg R A: The hallmarks of cancer. Cell 2000, 100(1):57-70.
Hentsch, B., Lyons, I., Li, R., Hartley, L,, Lints, T. J., Adams, J. M. and Harvey, R. P. (1996). Hlx homeo box gene is essential for an inductive tissue interaction that drives expansion of embryonic liver and gut. Genes Dev. 1, 70-79.
Higuchi, M., O'Brien, D., Kumaravelu, P., Lenny, N., Yeoh, E. J. and Downing, J. R. (2002), Expression of a conditional AMLI-ETO oncogene bypasses embryonic lethality and establishes a murine model of human t(8;21) acute myeloid leukemia. Cancer. Cell. 1, 63-74.
Horton, S. J., Grier, D. G., McGonigle, G. J., Thompson, A., Morrow, M., De Silva, I., Moulding, D. A., Kioussis, D., Lappin, T. R., Brady, H. J. and Williams, O. (2005). Continuous MLL-ENL expression is necessary to establish a “Hox Code” and maintain immortalization of hematopoietic progenitor cells. Cancer Res. 20, 9245-9252.
Huang da, W., Sherman, B. T. and Lempicki, R. A. (2009a). Bioinformatics enrichment tools: paths toward the comprehensive functional analysis of large gene lists. Nucleic Acids Res. 1, 1-13.
Huang da, W., Sherman. B. T. and Lempicki, R. A. (2009b), Systematic and integrative analysis of large gene lists using DAVID bioinformatics resources. Nat. Protoc. 1, 44-57.
Huntly, B. J., Shigematsu, H., Deguchi, K., Lee, B. H., Mizuno, S., Duclos, N., Rowan, R., Amaral, S., Curley, D., Williams, I. R., Akashi, K. and Gilliland, D. G. (2004). MOZ-TIF2, but not BCR-ABL, confers properties of leukemic stern cells to committed murine hematopoietic progenitors. Cancer. Cell. 6, 587-596.
Kawahara, M., Hori, T Chonabayashi, K., Oka, T., Sudol, M. and Uchiyama, T. (2008). Kpm/Lats2 is linked to chemosensitivity of leukemic cells through the stabilization of p73. Blood 9, 3856-3866.
Kawahara M, Pandolti A, Bartholdy B, Barreyro L, Will B, Roth M, Okoye-Okafor U C, Todorova T I, Figueroa M E, Melnick A, Mitsiades C S, Steidl U. H2.0-like homeobox regulates early hematopoiesis and promotes acute myeloid leukemia. Cancer Cell. 2012 Aug. 14;22(2):194-208.
Krivtsov, A V., Twomey, D., Feng, Z., Stubbs, M. C., Wang, Y., Faber, J., Levine, J. E., Wang, J., Hahn, W. C., Gilliland, D. G., Golub, T. R. and Armstrong, S. A. (2006). Transformation from committed progenitor to leukaemia stem cell initiated by MLL-AF9. Nature 7104, 818-822.
Kroon, E., Krosl, Thorsteinsdottir, U., Baban, S., Buchberg, A. M, and Sauvageau, G. (1998). Hoxa9 transforms primary bone marrow cells through specific collaboration with Meis la but not Pbx1b. EMBO J. 13, 3714-3725.
Kuo, M. L., Duncavage, E. J., Mathew, R., den Besten, W., Pei, D., Naeve, D., Yamamoto, T., Cheng, C., Sherr, C. J., and Roussel, M. F. (2003). Arf induces p53-dependent and -independent antiproliferative genes. Cancer Res. 63,1046-1053.
Leek, J. T., Johnson, W. E., Parker, H. S., Jaffe, A. E., and Storey, J. D. (2012). The sva package for removing batch effects and other unwanted variation in high-throughput experiments. Bioinformatics 28: 882-883.
Ley, T. J., Ding, L., Walter, M. J., McLellan, M. D., Lamprecht, T., Larson, D. E., Kandoth, C., Payton, J. E., Baty, J., Welch, J. et al . (2010). DNMT3A mutations in acute myeloid leukemia. N. Engl. J. Med. 25, 2424-2433.
Marcucci, G., Haferlach, T. and Dohner, H. (2011), Molecular genetics of adult acute myeloid leukemia: prognostic and therapeutic implications. J. Clin. Oncol. 5, 475-486.
Merico, ft. Isserlin, R., Stueker, O., Emili, A. and Bader, G. D. (2010). Enrichment map: a network-based method for gene-set enrichment visualization and interpretation. PLoS One 11, e13984.
Metzeler, K. H., Hummel, M., Bloomfield, C. D., Spiekermann, K., Braess, J., Sauerland, M. C., Heinecke, A., Radmacher, M., Marcucci, G., Whitman, S. P. et at (2008). An 86-probe-set gene-expression signature predicts survival in cytogenetically normal acute myeloid leukemia. Blood 10, 4193-4201.
Moens, C. B. and Selleri, (2006). Hox cofactors in vertebrate development. Dev. Biol. 2, 193-206.
Mootha, V. K., Lindgren, C. M., Eriksson, K. F., Subramanian, A., Sihag, S., Lehar, J., Puigserver, P., Carlsson, E., Ridderstrale, M., Laurila, E. et al. (2003). PGC-1alpha-responsive genes involved in oxidative phosphorylation are coordinately downregulated in human diabetes. Nat. Genet. 3, 267-273.
Novershtem, N., Subramanian, A., Lawton, L. N., Mak, R. H., Haining, W. N., McConkey, M. E., Habib, N., Yosef, N., Chang, C. Y., Shay, T., et al (2011). Densely interconnected transcriptional circuits control cell states in human hematopoiesis. Cell. 144, 296-309.
Ong, C. C., Jubb, A. M., Zhou, W., Haverty, P. M., Harris, A. L., Belvin, M., Friedman, L. S., Koeppen, H., and Hoeflich, K. P. (2011). p21-activated kinase 1: PAK'ed with potential. Oncotarget. 2, 491-496.
Pineault, N., Abramovich, C., Ohta, H. and Humphries, R. K. (2004), Differential and common leukemogenic potentials of multiple NUP98-Hox fusion proteins alone or with meis1. Mol. Cell. Biol, 5, 1907-1917.
Rosenbauer, F. and Tenen, D. G. (2007), Transcription factors in myeloid development: balancing differentiation with transfoi illation. Nat. Rev. Immunol, 2, 105-117.
Rosenbauer, F., Wagner, K., Kutok, J. L., Iwasaki, H., Le Beau, M. M., Okuno, Y., Akashi, K., Fiering, S. and Tenen, D. G. (2004). Acute myeloid leukemia induced by graded reduction of a lineage-specific transcription factor, PU.1. Nat. Genet. 6, 624-630.
Saeed, A. I., Bhagabati, N. K., Braisted, J. C., Liang, W., Sharov, V., Howe, E. A., Li, J., Thiagarajan, M., White, J. A., and Quackenbush, J. (2006). TM4 microarray software suite. Methods Enzymol, 134-193.
Sauvageau, G., Thorsteinsdottir, U., Eaves, C. J., Lawrence, H. J., Largman, C., Lansdorp, P. M. and Humphries, R. K. (1995). Overexpression of HOXB4 in hematopoietic cells causes the selective expansion of more primitive populations in vitro and in vivo. Genes Dev. 14, 1753-1765.
Sauvageau, G., Thorsteinsdottir, U., Hough, M. R., Hugo, P., Lawrence, H. J., Largman, C. and Humphries, R. K. (1997). Overexpression of HOXB3 in hematopoietic cells causes defective lymphoid development and progressive myeloproliferation. Immunity 1, 13-22.
Scholl, C., Bansal, D., Dohner, K., Eiwen, K., Huntly, B. J., Lee, B. H., Rucker, F. G., Schlenk, R. F., Bullinger, L., Dohner, H., Gilliland, D. G. and Frohling, S. (2007). The homeobox gene CDX2 is aberrantly expressed in most cases of acute myeloid leukemia and promotes leukemogenesis. J. Clin, Invest. 4, 1037-1048.
Somervaille, T. C. and Cleary, M. L. (2006). Identification and characterization of leukemia stem cells in marine MLL-Af9 acute myeloid leukemia. Cancer. Cell. 4, 257-268.
Steidl, U., Rosenbauer, F., Verhaak, R. G., Gu, X., Ebralidze, A., Otu, H. H., Klippel, S., Steidl, C., Bruns, I., Costa, D. B. et al . (2006). Essential role of Jun family transcription factors in PU.1 knockdown-induced leukemic stem cells. Nat. Genet, 11, 1269-1277.
Steidl, U., Steidl, C., Ebralidze, A Chapuy, B., Han, H. J., Will, B., Rosenbauer, F., Becker, A., Wagner, K., Koschmieder, S. et al . (2007). A distal single nucleotide polymorphism alters long-range regulation of the PU.1 gene in acute myeloid leukemia, J. Clin. Invest. 9, 2611-2620.
Subramanian, A., Tamayo, P., Mootha, V. K., Mukherjee, S., Ebert, B. L., Gillette, M. A., Paulovich, A., Pomeroy, S. L., Golub, T. R., Lander, E. S. and Mesirov, J. P. (2005). Gene set enrichment analysis: a knowledge-based approach for interpreting genome-wide expression profiles. Proc. Natl. Acad. Sci. U.S.A. 43, 15545-15550.
Thorsteinsdottir, U., Sauvageau, G., Hough, M. R,, Dragowska, W Lansdorp, P. M., Lawrence, H. J., Largman, C. and Humphries, R. K. (1997). Overexpression of HOXAIO in murine hematopoietic cells perturbs both myeloid and lymphoid differentiation and leads to acute myeloid leukemia. Mol. Cell. Biol. 1, 495-505.
Tomasson, M. H., Xiang, Z., Walgren, R., Zhao, Y., Kasai, Y., Miner, T., Ries, R. E., Lubman, O., Fremont, D. H., McLellan, M. D. et al . (2008). Somatic mutations and germline sequence variants in the expressed tyrosine kinase genes of patients with de novo acute myeloid leukemia. Blood 9, 4797-4808.
Will, B., Kawahara, M., Luciano, J. P., Bruns, I., Parekh, S., Erickson-Miller, C. L., Aivado, M. A., Verma, A. and Steidl, U. (2009). Effect of the nonpeptide thrombopoietin receptor agonist Eltrombopag on bone marrow cells from patients with acute myeloid leukemia and myelodysplastic syndrome. Blood 18, 3899-3908.
Wouters, B. J., Lowenberg, B., Erpelinck-Verschueren, C. A., van Putten, W. L., Valk, P. J. and Delwel, R. (2009). Double CEBPA mutations, but not single CEBPA mutations, define a subgroup of acute myeloid leukemia with a distinctive gene expression profile that is uniquely associated with a favorable outcome. Blood 13, 3088-3091.

What is claimed is:

1. A method of treating acute myeloid leukemia (AML), myelodysplastic syndrome (MDS), or a tumor having elevated expression of H2.0-like homeobox (HLX) and/or elevated expression of p21 protein (Cdc42/Rac)-activated kinase (PAK1) in a subject, the method comprising administering to the subject a PAK1 inhibitor in an amount effective to treat AML, MDS or the tumor having elevated expression of HLX and/or PAK1.

2. The method of claim 1, wherein the subject has AML.

3. The method of claim 1, wherein the subject has MDS.

4. The method of claim 1, wherein the subject has elevated expression of HLX.

5. The method of claim 1, wherein the subject has a tumor having elevated expression of HLX.

6. The method of claim 1, wherein the subject has a tumor having elevated expression of HLX and elevated expression of PAK1.

7. The method of claim 1, wherein the subject has a tumor having increased PAK1 activity.

8. The method of claim 1, wherein the PAK1 inhibitor is a pyrrole pyrazole compound or a pyrrolo[2,3-b]pyridine derivative.

9. The method of claim 1, wherein the PAK1 inhibitor is 1,1′-Disulfanediyldinaphthalen-2-ol.

10. The method of claim 1, wherein the PAK1 inhibitor is (S)-N-(2-(dimethylamino)-1-phenylethyl)-6,6-dimethyl-3-((2-methylthieno[3,2-d]pyrimidin-4-yl)amino)-4,6-dihydropyrrolo[3,4-c]pyrazole-5(1H)-carboxamide.

11. The method of claim 1, wherein the PAK1 inhibitor is a short hairpin nucleic acid.

12. The method of claim 11, wherein the short hairpin nucleic acid has the sequence CGATGAGAAATACCAGCACTA (SEQ ID NO:31) or GCGATCCTAAGAAGAAATATA (SEQ ID NO:32).

13. The method of claim 1, wherein the PAK1 inhibitor reduces proliferation of AML, MDS and/or tumor cells having elevated expression of HLX and/or PAK1.

14. The method of claim 1, wherein the PAK1 inhibitor induces apoptosis in AML, MDS and/or tumor cells having elevated expression of HLX and/or PAK1.

15. The method of claim 1, wherein the PAK1 inhibitor reduces colony formation of AML, MDS and/or tumor cells having elevated expression of HLX and/or PAK1.

16. The method of claim 1, wherein administration of the PAK1 inhibitor to the subject is effective to increase survival of the subject compared to untreated controls.

17. A method for screening for a candidate compound for treating a subject with acute myeloid leukemia (AML), myelodysplastic syndrome (MDS), and/or a tumor having elevated expression of H2.0-like homeobox (HLX) and/or elevated expression of p21 protein (Cdc42/Rac)-activated kinase (PAK1), the method comprising determining whether or not the compound inhibits PAK1, wherein a compound that inhibits PAK1 is a candidate compound for treating a subject with AML, MDS, and/or a tumor having elevated expression of HLX and/or elevated expression of PAK1.

18. The method of claim 17, wherein the method is carried out using an AML cell line.

19. The method of claim 18, wherein inhibition of PAK1 increases STAT4 expression, and/or reduces AKT1/2/3 serine 473 and/or STAT3 phosphorylation.


SEQUENCE LISTING (35 sequences)

SEQ ID NO: 1 - DNA, 23 bp, Artificial Sequence
  mouse H1x forward primer
ttcagcatcaattccaagacaca

SEQ ID NO: 2 - DNA, 22 bp, Artificial Sequence
  mouse H1x reverse primer
acctcttctccaggccttttct

SEQ ID NO: 3 - DNA, 22 bp, Artificial Sequence
  mouse Btgl1 foward primer
tcatctccaagttcctccgcac

SEQ ID NO: 4 - DNA, 22 bp, Artificial Sequence
  mouse Btgl1 reverse primer
caacggtaacctgatcccttgc

SEQ ID NO: 5 - DNA, 22 bp, Artificial Sequence
  mouse FoxO4 forward primer
tctacgaatggatggtccgcac

SEQ ID NO: 6 - DNA, 22 bp, Artificial Sequence
  mouse FoxO4 reverse primer
cttgctgtgcaaggacaggttg

SEQ ID NO: 7 - DNA, 22 bp, Artificial Sequence
  mouse Gadd45a forward primer
cctggaggaagtgctcagcaag

SEQ ID NO: 8 - DNA, 21 bp, Artificial Sequence
  mouse Gadd45a reverse primer
gtcgtcttcgtcagcagccag

SEQ ID NO: 9 - DNA, 22 bp, Artificial Sequence
  mouse Hdac7 forward primer
cgcctcaaactggataacggga

SEQ ID NO: 10 - DNA, 22 bp, Artificial Sequence
  mouse Hdac7 reverse primer
gcattggaggaatgcagctcgt

SEQ ID NO: 11 - DNA, 22 bp, Artificial Sequence
  mouse Pak1 foward primer
attgctccacgcccagaacaca

SEQ ID NO: 12 - DNA, 22 bp, Artificial Sequence
  mouse Pak1 reverse primer
aagcatctggcggagtggtgtt

SEQ ID NO: 13 - DNA, 19 bp, Artificial Sequence
  mouse Gapdh foward primer
ccagcctcgtcccgtagac

SEQ ID NO: 14 - DNA, 19 bp, Artificial Sequence
  mouse Gapdh reverse primer
gccttgactgtgccgttga

SEQ ID NO: 15 - DNA, 20 bp, Artificial Sequence
  mouse Trp63 forward primer
tgagccgtgagttcaatgag

SEQ ID NO: 16 - DNA, 20 bp, Artificial Sequence
  mouse Trp63 reverse primer
acctgtggtggctcataagg

SEQ ID NO: 17 - DNA, 20 bp, Artificial Sequence
  mouse RhoB forward primer
tattttcggaggcagaatgg

SEQ ID NO: 18 - DNA, 20 bp, Artificial Sequence
  mouse RhoB reverse primer
gatggccaagtccagttcat

SEQ ID NO: 19 - DNA, 20 bp, Artificial Sequence
  mouse Zfp361 forward primer
cccctaccttggacaactca

SEQ ID NO: 20 - DNA, 20 bp, Artificial Sequence
  mouse Zfp361 reverse primer
aagagatggggtggagaggt

SEQ ID NO: 21 - DNA, 20 bp, Artificial Sequence
  mouse Fyn forward primer
ggaaaaaggatccggaagag

SEQ ID NO: 22 - DNA, 20 bp, Artificial Sequence
  mouse Fyn reverse primer
gcaggctctcacaggttttc

SEQ ID NO: 23 - DNA, 22 bp, Artificial Sequence
  human PAK1 forward primer
gtgaaggctgtgtctgagactc

SEQ ID NO: 24 - DNA, 23 bp, Artificial Sequence
  human PAK1 reverse primer
ggaagtggttcaatcacagaccg

SEQ ID NO: 25 - DNA, 22 bp, Artificial Sequence
  human BTG1 forward primer
catctccaagtttctccgcacc

SEQ ID NO: 26 - DNA, 23 bp, Artificial Sequence
  human BTG1 reverse primer
gcgaatacaacggtaacccgatc

SEQ ID NO: 27 - DNA, 26 bp, Artificial Sequence
  short hairpin sequence for luciferase target
gtgcgttgttagtactaatcctattt

SEQ ID NO: 28 - DNA, 29 bp, Artificial Sequence
  short hairpin sequence for mouse H1x target
ggcgcagaaggacaaggacaaggaagcgg

SEQ ID NO: 29 - DNA, 22 bp, Artificial Sequence
  non-silencing control
acctccaccctcactctgccat

SEQ ID NO: 30 - DNA, 19 bp, Artificial Sequence
  short hairpin sequence for human HLX target
ccctatgctgtgctcacga

SEQ ID NO: 31 - DNA, 21 bp, Artificial Sequence
  short hairpin sequence for human PAK1 #1
cgatgagaaataccagcacta

SEQ ID NO: 32 - DNA, 21 bp, Artificial Sequence
  short hairpin sequence for human PAK1 #2
gcgatcctaagaagaaatata

SEQ ID NO: 33 - DNA, 2308 bp, Homo sapiens
aaaactttgggagtttttagagacgagtttttttttttttctattacttttccccccccctaactaacggactattattgttgttgttttaaatttagctcttagggcttagctatttgggttttcttgcggtgtccggctcccgtctccctggctcccccgcccgccctgcggccccagcgcccctcgctctcatccagcccgcgaggagtgcgggcgccgcgccgcctttaaagcgaggccagggagcgaggcggtgaccggccgagatccggccctcgcctcctccctcggtggcgctagggctcccggcctctcttcctcagtgcgggcggagaagcgaaagcggatcgtcctcggctgccgccgccttctccgggactcgcgcgcccctccccgcgcgcccacccacccagtccggctggactgcggcagccgcgcggctcaccccggcaggatgttcgcagccgggctggctcccttctacgcctccaacttcagcctctggtcggccgcttactgctcctcggccggcccaggcggctgctccttccccttggaccccgccgccgtcaaaaagccctccttctgcatcgcagacattctgcacgccggcgtgggggatctgggggcggccccggagggcctggcaggggcctcggccgccgccctcaccgcgcacttgggctcggttcacccgcacgcctctttccaagcggcggccagatccccgcttcgacccaccccagtggtggcgccctccgaagtcccggctggcttcccgcagcggctgtctccgctctcagccgcctaccaccaccatcacccgcaacaacaacagcagcagcaacagccgcagcagcaacagcctccgcctccgccccgggctggcgccctgcagcccccggcctcggggacgcgagtggttccgaacccccaccacagtggctctgccccggccccctccagcaaagacctcaaatttggaattgaccgcattttatctgcagaatttgacccaaaagtcaaagaaggcaacacgctgagagatctcacttccctgctaaccggtgggcggcccgccggggtgcacctctcaggcctgcagccctcggccggccagttcttcgcatctctagatcccattaacgaggcttctgcaatcctgagtcccttaaactcgaacccaagaaattcagttcagcatcagttccaagacacgtttccaggtccctatgctgtgctcacgaaggacaccatgccgcagacgtacaaaaggaagcgttcatggtcgcgcgctgtgttctccaacctgcagaggaaaggcctggagaaaaggtttgagattcagaagtacgtgaccaagccggaccgaaagcagctggcggcgatgctgggcctcacggacgcacaggtgaaggtgtggttccagaaccggcggatgaagtggcggcactccaaggaggcccaggcccaaaaggacaaggacaaggaggctggcgagaagccatcaggtggagccccggctgcggatggcgagcaggacgagaggagccccagccgttctgaaggcgaggctgagagcgagagcagcgactccgagtccctggacatggcccccagcgacacggagcggactgaggggagtgagcgttctctgcaccaaacaacagttattaaggccccggtcactggcgccctcattaccgccagcagtgctgggagtggtgggagcagcggcggcggcggcaatagtttcagcttcagcagcgccagcagtcttagtagcagcagcaccagtgcgggttgcgccagcagccttggcggcggcggcgcctcggagcttctccctgcaacacagcccacagccagcagcgctcccaaaagccccgagccagcccaaggcgcgcttggctgcttatagactgtactagggcggaggggatccgggccttgcgtgcagcctcccaaccatgggctgggttttgtgcttactgtatgttggcgacttggtagggcaggagacgcagcgtggagcctacctcccgacattcacgcttcgccccacgctgctccgactggctgcagcggacactgcccaaagcagaggggagtctcagtgtcctgctagccagccgaacacttctctccggaagcaggctggttcgactgtgaggtgtttgactaaactgtttctctgactcgccccagaggtcgtggctcaaaggcacttaggacgccttaaatttgtaaataaaatgtttactacggtttgtaaaaaaaaaaaaaaaaaaaaaaaaaaaaaaaa

SEQ ID NO: 34 - protein, 488 aa, Homo sapiens
MFAAGLAPFYASNFSLWSAAYCSSAGPGGCSFPLDPAAVKKPSFCIADILHAGVGDLGAAPEGLAGASAAALTAHLGSVHPHASFQAAARSPLRPTPVVAPSEVPAGFPQRLSPLSAAYHHHHPQQQQQQQQPQQQQPPPPPRAGALQPPASGTRVVPNPHHSGSAPAPSSKDLKFGIDRILSAEFDPKVKEGNTLRDLTSLLTGGRPAGVHLSGLQPSAGQFFASLDPINEASAILSPLNSNPRNSVQHQFQDTFPGPYAVLTKDTMPQTYKRKRSWSRAVFSNLQRKGLEKRFEIQKYVTKPDRKQLAAMLGLTDAQVKVWFQNRRMKWRHSKEAQAQKDKDKEAGEKPSGGAPAADGEQDERSPSRSEGEAESESSDSESLDMAPSDTERTEGSERSLHQTTVIKAPVTGALITASSAGSGGSSGGGGNSFSFSSASSLSSSSTSAGCASSLGGGGASELLPATQPTASSAPKSPEPAQGALGCL

SEQ ID NO: 35 - protein, 545 aa, Homo sapiens
MSNNGLDIQDKPPAPPMRNTSTMIGAGSKDAGTLNHGSKPLPPNPEEKKKKDRFYRSILPGDKTNKKKEKERPEISLPSDFEHTIHVGFDAVTGEFTGMPEQWARLLQTSNITKSEQKKNPQAVLDVLEFYNSKKTSNSQKYMSFTDKSAEDYNSSNALNVKAVSETPAVPPVSEDEDDDDDDATPPPVIAPRPEHTKSVYTRSVIEPLPVTPTRDVATSPISPTENNTTPPDALTRNTEKQKKKPKMSDEEILEKLRSIVSVGDPKKKYTRFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLEMDVEKRGSAKELLQHQFLKIAKPLSSLTPLIAAAKEATKNNH